SEQUENCE LISTING (19 sequences)

SEQ ID NO: 1 - protein, 11 aa, Artificial Sequence
  Synthetic Peptide
YGRKKRRQRRR

SEQ ID NO: 2 - protein, 12 aa, Artificial Sequence
  Synthetic Peptide
RRQRRTSKLMKR

SEQ ID NO: 3 - protein, 27 aa, Artificial Sequence
  Synthetic Peptide
GWTLNSAGYLLGKINLKALAALAKKIL

SEQ ID NO: 4 - protein, 33 aa, Artificial Sequence
  Synthetic Peptide
KALAWEAKLAKALAKALAKHLAKALAKALKCEA

SEQ ID NO: 5 - protein, 16 aa, Artificial Sequence
  Synthetic Peptide
RQIKIWFQNRRMKWKK

SEQ ID NO: 6 - protein, 9 aa, Artificial Sequence
  Synthetic Peptide
RKKRRQRRR

SEQ ID NO: 7 - protein, 8 aa, Artificial Sequence
  Synthetic Peptide
RKKRRQRR

SEQ ID NO: 8 - protein, 11 aa, Artificial Sequence
  Synthetic Peptide
YARAAARQARA

SEQ ID NO: 9 - protein, 11 aa, Artificial Sequence
  Synthetic Peptide
THRLPRRRRRR

SEQ ID NO: 10 - protein, 11 aa, Artificial Sequence
  Synthetic Peptide
GGRRARRRRRR

SEQ ID NO: 11 - protein, 5 aa, Artificial Sequence
  Synthetic Peptide
  MISC_FEATURE (1)..(5): This stretch of residues may be present 1 to 50 times.
GSGGS

SEQ ID NO: 12 - protein, 5 aa, Artificial Sequence
  Synthetic Peptide
  MISC_FEATURE (1)..(1): This residue may be present 1 to 20 times.
  MISC_FEATURE (1)..(5): This stretch of residues may be present 1 to 20 times.
  MISC_FEATURE (2)..(2): This residue may be present 1 to 20 times.
  MISC_FEATURE (3)..(3): This residue may be present 1 to 20 times.
  MISC_FEATURE (4)..(4): This residue may be present 1 to 20 times.
  MISC_FEATURE (5)..(5): This residue may be present 1 to 20 times.
GSGSG

SEQ ID NO: 13 - protein, 4 aa, Artificial Sequence
  Synthetic Peptide
  MISC_FEATURE (1)..(4): This stretch of residues may be present 1 to 50 times.
  MISC_FEATURE (4)..(4): This residue may be present 1 to 20 times.
GGGS

SEQ ID NO: 14 - protein, 4 aa, Artificial Sequence
  Synthetic Peptide
GGSG

SEQ ID NO: 15 - protein, 5 aa, Artificial Sequence
  Synthetic Peptide
GGSGG

SEQ ID NO: 16 - protein, 5 aa, Artificial Sequence
  Synthetic Peptide
GSGGG

SEQ ID NO: 17 - protein, 5 aa, Artificial Sequence
  Synthetic Peptide
GGGSG

SEQ ID NO: 18 - protein, 5 aa, Artificial Sequence
  Synthetic Peptide
GSSSG

SEQ ID NO: 19 - protein, 5 aa, Artificial Sequence
  Synthetic Peptide
  MISC_FEATURE (1)..(5): This strech of residues may be present 1 to 50 times.
GGGGS

(19) United States
(12) Patent Application Publication
(10) Pub. No.: US 2019/0307849 A1
(43) Pub. Date: Oct. 10, 2019

(54) METHODS OF USING INTERLEUKIN-10 FOR TREATING DISEASES AND DISORDERS

(71) Applicant: ARMO BioSciences, Inc., Redwood City, CA (US)

(72) Inventor: Martin Oft, Palo Alto, CA (US)

(21) Appl. No.: 15/576,239

(22) PCT Filed: May 26, 2016
(86) PCT No.: PCT/US2016/034404

§ 371 Date: Nov. 21, 2017

Related U.S. Application Data
(60) Provisional application No. 62/168,523, filed on May 29, 2015.

(51) Int. Cl. A61K 38/20; A61K 9/00; C07K 14/54; A61K 38/38; A61K 39/395; C07K 16/28; A61P 35/00

(52) U.S. Cl. CPC A61K 38/2066; A61K 9/0019; A61K 38/2046; C07K 14/5428; A61K 38/385; C07K 2317/24; C07K 16/2818; A61P 35/00; C07K 2319/30; C07K 2317/21; A61K 39/39541

(57) ABSTRACT

Methods of treating subjects having a cancer-related, immune-related, or viral-related disease, disorder or condition via administration of an IL-10 agent, including pegylated IL-10, and an IL-7 agent are provided, as are methods of identifying subjects that may be responsive to treatment with an IL-10 agent.

### CROSS-REFERENCE TO RELATED APPLICATION

The present application claims priority benefit of U.S. provisional application Ser. No. 62/168,523, filed May 29, 2015, which application is incorporated herein by reference in its entirety.

### FIELD OF THE INVENTION

This invention relates to methods of using IL-10 agents as monotherapy and in combination with other agents in the treatment or prevention of a diverse array of diseases and disorders, including cancers and immune-related disorders.

### INTRODUCTION

The cytokine interleukin-10 (IL-10) is a pleiotropic cytokine that regulates multiple immune responses through actions on T cells, B cells, macrophages, and antigen presenting cells (APC). IL-10 can suppress immune responses by inhibiting expression of IL-1 1α, IL-1β, IL-6, IL-8, TNF-α, GM-CSF and G-CSF in activated monocytes and activated macrophages, and it also suppresses IFN-γ production by NK cells. Although IL-10 is predominantly expressed in macrophages, expression has also been detected in activated T cells, B cells, mast cells, and monocytes. In addition to suppressing immune responses, IL-10 exhibits immuno-stimulatory properties, including stimulating the proliferation of IL-2- and IL-4-treated thymocytes, enhancing the viability of B cells, and stimulating the expression of MHC class II.

Human IL-10 is a homodimer that becomes biologically inactive upon disruption of the non-covalent interactions between the two monomer subunits. Data obtained from the published crystal structure of IL-10 indicates that the functional dimer exhibits certain similarities to IFN-γ (Zdanov et al, (1995) Structure (Lond) 3:591-601).

As a result of its pleiotropic activity, IL-10 has been linked to a broad range of diseases, disorders and conditions, including inflammatory conditions, immune-related disorders, fibrotic disorders, metabolic disorders and cancer. Clinical and pre-clinical evaluations with IL-10 for a number of such diseases, disorders and conditions have solidified its therapeutic potential. Moreover, pegylated IL-10 has been shown to be more efficacious than non-pegylated IL-10 in certain therapeutic settings.

### SUMMARY

The present disclosure contemplates methods of using IL-10, modified (e.g., pegylated) IL-10, and associated agents described herein, and compositions thereof, in combination with an IL-7 agent and/or another agent(s), for the treatment and/or prevention of diseases, disorders and conditions associated with cancer (e.g., solid tumors), inflammation, the immune system, and infection (e.g., viral infection). The present disclosure also contemplates methods relating to, for example, identifying subjects having or suspected of having a cancer-related disease, disorder or condition who are likely to be responsive to treatment with an IL-10 agent.

As described hereafter, oncology patients that have shown a beneficial response, including stable disease, to IL-10 therapy have exhibited serum IL-7 levels higher than non-responders to IL-10 treatment. More particularly, in response to treatment with PEG-IL-10, serum IL-7 levels were significantly up-regulated in patients having disease stabilization (SD; stable disease) or tumor shrinkage (PR; partial response); although serum IL-7 levels were also up-regulated in patients having progressive disease (PD), the elevation was less pronounced. Thus, the use of IL-7 as an adjuvant to IL-10 therapy in such responsive patients may provide additional therapeutic benefit. Such combinations provide the opportunity for additive or synergistic effects in the treatment and/or prevention of the diseases, disorders and conditions described herein. Moreover, such combination therapy often allows for reductions in the amounts and/or frequencies of administration of an IL-10 agent (e.g., PEG-IL-10) and the other agent(s) (e.g., an IL-7 agent) in which it is combined, which can result in any adverse effects being minimized or obviated.

The terms “IL-7”, “IL-7 polypeptide(s)”, “IL-7 molecule(s)”, “IL-7 agent(s)” and the like are intended to be construed broadly and include, for example, human and non-human IL-7-related polypeptides, including homologs, variants (including muteins), and fragments thereof, as well as IL-7 polypeptides having, for example, a leader sequence, and modified versions of the foregoing. In further particular embodiments, the IL-7 agents are agonists. In certain embodiments, the IL-7 agent is mature human IL-10. In other embodiments, the IL-7 agent is a variant of mature human IL-7, wherein the variant exhibits activity comparable to the activity of mature human IL-7. The present disclosure also contemplates nucleic acid molecules encoding the foregoing.

The present disclosure also contemplates methods and models comprising the use of IL-7 for optimizing dosing regimens for IL-10 agents. Methods and models for identifying specific patients and patient populations that are optimally suited for the therapies described herein are also contemplated, as are methods of monitoring the responsiveness of such patients to the therapies described herein. In such methods and models, IL-7 can be used as an indicator (e.g., a surrogate marker) of a subject that who is likely to respond to IL-10 therapy, the effectiveness of IL-10 therapy, etc.

As discussed further hereafter, human IL-10 is a homodimer, and each monomer comprises 178 amino acids, the first 18 of which comprise a signal peptide. Particular embodiments of the present disclosure comprise mature human IL-10 polypeptides lacking the signal peptide (see, e.g., U.S. Pat. No. 6,217,857), or mature human PEG-IL-10. In further particular embodiments, the IL-10 agent is a variant of mature human IL-10. The variant can exhibit activity less than, comparable to, or greater than the activity of mature human IL-10; in certain embodiments the activity is comparable to or greater than the activity of mature human IL-10.

Certain embodiments of the present disclosure contemplate modification of IL-10 in order to enhance one or more properties (e.g., pharmacokinetic parameters, efficacy, etc.). Such IL-10 modifications include pegylation, glycosylation, albumin (e.g., human serum albumin (HSA)) conjugation and fusion, and hesylation. In particular embodiments, IL-10 is pegylated. In further embodiments, modification of IL-10 does not result in a therapeutically relevant, detrimental effect on immunogenicity, and in still further embodiments modified IL-10 is less immunogenic than unmodified IL-10. The terms “IL-10”, “IL-10 polypeptide(s)”, “IL-10 molecule(s)”, “IL-10 agent(s)” and the like are intended to be construed broadly and include, for example, human and non-human IL-10-related polypeptides, including homologs, variants (including muteins), and fragments thereof, as well as IL-10 polypeptides having, for example, a leader sequence (e.g., the signal peptide), and modified versions of the foregoing. In further particular embodiments, the terms “IL-10”, “IL-10 polypeptide(s), “agent(s)” are agonists. Particular embodiments relate to pegylated IL-10, which is also referred to herein as “PEG-IL-10”. In certain embodiments, the IL-10 agent is mature human IL-10. In other embodiments, the IL-10 agent is a variant of mature human IL-10, wherein the variant exhibits activity comparable to the activity of mature human IL-10. The present disclosure also contemplates nucleic acid molecules encoding the foregoing.

Particular embodiments of the present disclosure relate to methods of treating or preventing a disease, disorder or condition in a subject (e.g., a human), comprising administering to the subject therapeutically effective amounts of an IL-7 agent and an IL-10 agent; wherein the disease disorder or condition is a cancer-related disease, disorder or condition; an immune-related disease, disorder or condition; or a viral-related disease, disorder or condition.

Other embodiments of the present disclosure contemplate methods of treating or preventing a disease, disorder or condition in a subject, comprising administering to the subject therapeutically effective amounts of an IL-7 agent and an IL-10 agent, wherein the amount of the IL-10 agent is sufficient to maintain a mean IL-10 serum trough concentration over a period of time, and wherein the disease disorder or condition is a cancer-related disease, disorder or condition; an immune-related disease, disorder or condition; or a viral-related disease, disorder or condition. In particular embodiments, the mean IL-10 serum trough concentration is at least 1.0 ng/mL. In some embodiments, the mean IL-10 serum trough concentration is maintained for at least 90% of the period of time.

Other embodiments are drawn to methods of treating or preventing a cancer-related, immune-related, or viral-related disease, disorder or condition in a subject, comprising administering to the subject an IL-10 agent in an amount sufficient to maintain a mean IL-10 serum trough concentration from 1 pg/mL to 10.0 ng/mL. In some embodiments, the mean IL-10 serum trough concentration of from 1.0 pg/mL to 10.0 ng/mL is maintained for at least 95% of the period of time.

In some embodiments of the present disclosure, the mean IL-10 serum trough concentration is in the range of from 1.0 pg/mL to 100 pg/mL; from 0.1 ng/mL to 1.0 ng/mL; from 1.0 ng/mL to 10 ng/mL; from 0.5 ng/mL to 5.0 ng/mL; from 0.75 ng/mL to 1.25 ng/mL or from 0.9 ng/mL to 1.1 ng/mL. In particular embodiments of the present disclosure, the mean IL-10 serum trough concentration is at least 1.25 ng/mL, at least 1.5 ng/mL, at least 1.6 ng/mL, at least 1.7 ng/mL, at least 1.8 ng/mL, at least 1.85 ng/mL, at least 1.9 ng/mL, at least 1.95 ng/mL, at least 1.97 ng/mL, and least 1.98 ng/mL, at least 1.99 ng/mL, at least 2.0 ng/mL or greater than 2 ng/mL.

In further embodiments, the aforementioned period of time is at least 12 hours, at least 24 hours, at least 48 hours, at least 72 hours, at least 1 week, at least 2 weeks, at least 3 weeks, at least 1 month, at least 6 weeks, at least 2 months, at least 3 months, at least 6 months, at least 9 months, or greater than 12 months.

In particular embodiments of the present disclosure, the mean IL-10 serum trough concentration is maintained for at least 85% of the period of time, at least 90%, at least 96%, at least 98%, at least 99% or 100% of the period of time.

It is envisaged that a dosing regimen sufficient to maintain a desired steady state serum trough concentration (e.g., 1 ng/mL) can result in an initial serum trough concentration that is higher than the desired steady state serum trough concentration. Because of the pharmacodynamic and pharmacokinetic characteristics of IL-10 in a mammalian subject, an initial trough concentration (achieved, for example, through the administration of one or more loading doses followed by a series of maintenance doses) gradually but continually decreases over a period of time even when the dosing parameters (amount and frequency) are kept constant. After that period to time, the gradual but continual decrease ends and a steady state serum trough concentration is maintained.

By way of example, parenteral administration (e.g., SC and IV) of ˜0.1 mg/kg/day of an IL-10 agent (e.g., mIL-10) to a mouse (e.g., a C57BL/6 mouse) is required to maintain a steady state serum trough concentration of 2.0 ng/mL. However, that steady state serum trough concentration cannot be achieved until approximately 30 days after initiation of dosing at 0.1 mg/kg/day (and also after any loading dose(s)). Rather, after an initial serum trough concentration has been achieved (e.g., 2.5 ng/mL), that concentration gradually but continually decreases over the course of, for example, the approximately 30-day period, after which time the desired steady state serum trough concentration (2.0 ng/mL) is maintained. One of skill in the art will be able to determine the dose needed to maintain the desired steady state trough concentration using, for example, ADME and patient-specific parameters.

The present disclosure contemplates methods wherein the IL-10 agent comprises at least one modification to form a modified IL-10 agent, wherein the modification does not alter the amino acid sequence of the IL-10 agent. In some embodiments, the modified IL-10 agent is a PEG-IL-10 agent. The PEG-IL-10 agent can comprise at least one PEG molecule covalently attached to at least one amino acid residue of at least one subunit of IL-10 or comprise a mixture of mono-pegylated and di-pegylated IL-10 in other embodiments. The PEG component of the PEG-IL-10 agent can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa.

In some embodiments, the modified IL-10 agent comprises at least one Fc fusion molecule, at least one serum albumin (e.g., HSA or BSA), an HSA fusion molecule or an albumin conjugate. In additional embodiments, the modified IL-10 agent is glycosylated, is hesylated, or comprises at least one albumin binding domain. Some modified IL-10 agents can comprise more than one type of modification. In particular embodiments, the modification is site-specific. Some embodiments comprise a linker. Modified IL-10 agents are discussed in detail hereafter.

Particular embodiments of the present disclosure are drawn to methods of treating or preventing a cancer-related disease, disorder or condition in a subject (e.g., a human). In some embodiments, the cancer-related disease, disorder of condition is a solid tumor or a hematological disorder. Examples of cancer-related diseases, disorders or conditions contemplated herein include melanoma, lung cancer, renal cancer, breast cancer, colon cancer, head and neck cancer, leukemia, and lymphoma. Other cancers are set forth hereafter.

Further particular embodiments of the present disclosure are drawn to methods of treating or preventing an immune-related, inflammatory-related, or viral-related disease, disorder or condition in a subject (e.g., a human). In a particular embodiment, the viral-related disease, disorder of condition is HIV. Examples of immune-related, inflammatory-related and viral-related diseases, disorders or conditions are set forth hereafter.

The present disclosure contemplates administration of the IL-10 agents and the IL-7 agents via any suitable route of administration. By way of example, an IL-10 agent and/or an IL-7 agent can be administered parenterally (e.g., subcutaneously or intravenously). In certain embodiments, the IL-10 agent and the IL-7 agent are administered simultaneously, while in other embodiments they are administered sequentially. It is to be understood that the IL-10 agent, the IL-7 agent, and any additional agents (e.g., prophylactic or therapeutic agents) may be administered by different routes; for example, the IL-10 agent may be administered subcutaneously and an additional agent may be administered orally. In some embodiments of the present disclosure, an additional agent is a chemotherapeutic agent, an agent for the treatment or prevention of an immune or inflammatory disorder, or an agent for the treatment or prevention of a viral disorder. Examples of additional agents (e.g., prophylactic or therapeutic agents) that may be used in the methods described herein are described hereafter.

Still further embodiments of the present disclosure contemplate pharmaceutical compositions comprising a therapeutically effective amount of an IL-10 agent (e.g., PEG-IL-10) and a therapeutically effective amount of an IL-7 agent, and a pharmaceutically acceptable diluent, carrier or excipient (e.g., an isotonic injection solution). In some embodiments, the pharmaceutical composition is suitable for human administration. Embodiments contemplated herein include such pharmaceutical compositions further comprising at least one additional prophylactic or therapeutic agent (e.g., a chemotherapeutic agent).

Certain embodiments of the present disclosure contemplate a sterile container that contains one of the above-mentioned components (e.g., a PEG-IL-10 agent) and optionally one or more additional components (e.g., an IL-7 agent). By way of example, but not limitation, the sterile container can be a syringe or a vial. In still further embodiments, the sterile container is one component of a kit; the kit can also contain, for example, a second sterile container that comprises an IL-7 agent and/or at least one prophylactic or therapeutic agent, examples of which are set forth herein.

As described further herein, PEG-IL-10 treatment at therapeutically effective doses induced up-regulation of several inflammatory cytokines, including IL-7, GM-CSF, IL-4 and IFNγ. In particular, IL-7 was significantly up-regulated in patients having disease stabilization or tumor shrinkage. Thus, serum concentration levels of IL-7 can be used as a biomarker to indicate that a therapeutic serum concentration of IL-10 has been achieved and/or that the PEG-IL-10 dose administered to achieve that serum concentration was efficacious.

Although the discussion hereafter focuses on the use of IL-7 as a biomarker, it is to be understood that other inflammatory cytokines described herein (e.g., GM-CSF, IL-4 and IFNy) may also be useful.

The present disclosure contemplates methods of identifying a subject having or suspected of having a cancer-related disease, disorder or condition who is likely to be responsive to treatment with an IL-10 agent, comprising administering the IL-10 agent to the subject having or suspected of having a cancer-related disease, disorder or condition; obtaining a test sample from the subject; determining the level of IL-7 in the test sample; and comparing the level of IL-7 in the test sample to a reference standard; wherein a subject having a higher level of IL-7 in the test sample than in the reference standard is likely to be responsive to treatment with the IL-10 agent.

Other embodiments of the present disclosure are directed to methods of predicting the responsiveness of a subject having or suspected of having a cancer-related disease, disorder or condition to an IL-10 agent, comprising administering the IL-10 agent to the subject having or suspected of having a cancer-related disease, disorder or condition;obtaining a test sample from the subject; determining the level of IL-7 in the test sample, and comparing the level of IL-7 in the test sample to a reference standard; wherein a subject having a level of IL-7 in the test sample that is greater than the reference standard by a target amount is likely to be responsive to treatment with the IL-10 agent.

In still further embodiments, contemplated herein are methods of monitoring the efficacy of a treatment regimen of a cancer-related disease, disorder or condition in a subject with an IL-10 agent, comprising administering the IL-10 agent to the subject having or suspected of having a cancer-related disease, disorder or condition; obtaining a test sample from the subject; determining the level of IL-7 in the test sample; and comparing the level of IL-7 in the test sample to the level of IL-7 in a reference standard; wherein a decrease in the level of IL-7 in the test sample compared to the reference standard is indicative of the efficacy of the IL-10 agent in treating the cancer-related disease, disorder or condition in the subject.

In particular embodiments, in the methods described above the reference standard is the level of IL-7 in a control sample obtained from the subject prior to administering the IL-10 agent to the subject, wherein the control sample is from the same source as the test sample. In other embodiments, in the methods described above the reference standard is the level of IL-7 determined from one or more control samples obtained from one or more control subjects having a cancer-related disease, disorder or condition, wherein the reference standard is from the same source as the one or more control samples. The source can be any biological fluid and the like, including serum.

According to the present disclosure, in some of the foregoing methods the level of IL-7 after administration of the IL-10 agent is at least 5% greater than the reference standard. In other embodiments, the level of IL-7 after administration of the IL-10 agent is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% greater than the reference standard. In particular embodiments of the present disclosure, the level of IL-7 after administration of the IL-10 agent is at least 5% less than the reference standard. In other embodiments, the level of IL-7 after administration of the IL-10 agent is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% less than the reference standard.

The present disclosure also contemplates methods of treating a subject having a cancer-related disease, disorder or condition, comprising obtaining a test sample from the subject, determining the level of IL-7 in the test sample, identifying the subject as likely to be responsive to an IL-10 agent if the level of IL-7 in the test sample is higher than a reference standard, and administering a therapeutically effective amount of the IL-10 agent to the subject identified as likely to be responsive to the IL-10 agent. Particular embodiments further comprise administering the IL-10 agent to the subject prior to obtaining a test sample from the subject, and comparing the level of IL-7 in the test sample after administering the IL-10 agent with a reference standard; wherein a level of IL-7 in the test sample after administering the IL-10 agent that is higher than the reference standard indicates that the subject is in need of an additional amount of and IL-10 agent to treat the cancer-related disease, disorder or condition; and administering an additional amount of the IL-10 agent to the subject.

In particular embodiments of the present disclosure, the reference standard is the level of IL-7 in a control sample obtained from the subject prior to administering the IL-10 agent to the subject, wherein the control sample is from the same source as the test sample. In other embodiments, the reference standard is the level of IL-7 determined from one or more control samples obtained from one or more control subjects having a cancer-related disease, disorder or condition, wherein the reference standard is from the same source as the one or more control samples. In still other embodiments, the reference standard is the level of IL-7 determined from one or more control samples obtained from one or more control subjects that do not have a cancer-related disease, disorder or condition, wherein the reference standard is from the same source as the one or more control samples. The source can be any biological fluid and the like, including serum.

In some embodiments of the foregoing methods, the level of IL-7 in the test sample is 1.5-fold to 10-fold of the reference standard. In other embodiments, the level of IL-7 in the test sample is 0.5-fold to 25-fold of the reference standard, 0.5-fold to 20-fold of the reference standard, 0.5-fold to 15-fold of the reference standard, 1.0-fold to 25-fold of the reference standard, 1.0-fold to 20-fold of the reference standard, 1.0-fold to 15-fold of the reference standard, 1.5-fold to 25-fold of the reference standard, 1.5-fold to 20-fold of the reference standard, 1.5-fold to 15-fold of the reference standard, and so forth.

The present disclosure contemplates embodiments wherein the IL-10 agent is mature human IL-10. In other embodiments, the IL-10 agent is a variant of mature human IL-10, wherein the variant exhibits activity comparable to the activity of mature human IL-10.

In certain embodiments of the present disclosure, the cancer-related disease, disorder or condition is a solid tumor or a hematological disorder. The cancer-related disease, disorder or condition is melanoma, lung cancer, renal cancer colon cancer, head and neck cancer and breast cancer, in some embodiments, while in others it is leukemia or lymphoma. Other cancer-related diseases, disorders and conditions are described herein.

The present disclosure contemplates methods wherein the IL-10 agent comprises at least one modification to form a modified IL-10 agent, wherein the modification does not alter the amino acid sequence of the IL-10 agent. In some embodiments, the modified IL-10 agent is a PEG-IL-10 agent. The PEG-IL-10 agent can comprise at least one PEG molecule covalently attached to at least one amino acid residue of at least one subunit of IL-10 or comprise a mixture of mono-pegylated and di-pegylated IL-10 in other embodiments. The PEG component of the PEG-IL-10 agent can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa.

In some embodiments, the modified IL-10 agent comprises at least one Fc fusion molecule, at least one serum albumin (e.g., HSA or BSA), an HSA fusion molecule or an albumin conjugate. In additional embodiments, the modified IL-10 agent is glycosylated, is hesylated, or comprises at least one albumin binding domain. Some modified IL-10 agents can comprise more than one type of modification. In particular embodiments, the modification is site-specific. Some embodiments comprise a linker. Modified IL-10 agents are discussed in detail hereafter.

In further embodiments, the present disclosure contemplates methods wherein the level of IL-7 is measured by determining the mRNA levels of IL-7. In other embodiments, the level of IL-7 is measured by determining the cDNA levels of IL-7. In still other embodiments, the level of IL-7 is measured by determining the protein levels of IL-7; in some embodiments, the IL-7 protein is isolated from the sample using liquid chromatography-tandem mass spectrometry (LCMS/MS).

Also contemplated herein are methods of determining the level of IL-7 in a sample comprising contacting proteins within the sample with a first antibody that immunospecifically binds to IL-7 protein. Further embodiments of the present disclosure comprise contacting the IL-7 protein bound to the first antibody with a second antibody having a detectable label, wherein the second antibody immunospecifically binds to the IL-7 protein, and wherein the second antibody immunospecifically binds to a different epitope on the IL-7 protein than the first antibody; detecting the presence of second antibody bound to the proteins; and determining the amount of IL-7 based on the amount of detectable label in the second antibody. According to the present disclosure, methods are also contemplated comprising contacting the IL-7 protein bound to the first antibody with a second antibody with a detectable label, wherein the second antibody immunospecifically binds to the first antibody; detecting the presence of second antibody bound to the proteins; and determining the level of the IL-7 protein based on the amount of detectable label in the second antibody.

The skilled artisan will envisage other embodiments in view of the teachings set forth herein, and the present disclosure contemplates the inclusion of such embodiments.

### BRIEF DESCRIPTION OF THE DRAWINGS

FIG. 1 indicates that treatment of subjects with therapeutically effective doses of PEG-hIL-10 induced a significant up-regulation of the cytokines IL-7, GM-CSF, IL-4 and IFNγ.

FIG. 2 indicates that treatment of subjects with therapeutically effective doses of PEG-hIL-10 resulted in significant up-regulation of IL-7, particularly in patients having disease stabilization (SD) or tumor shrinkage (PR). Up-regulation of FasL was also observed in a similar manner.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Overview

In the oncology setting, data presented and discussed hereafter support the use of an IL-7 agent in combination with IL-10 therapy. In response to treatment with PEG-IL-10, oncology patients' serum IL-7 levels were significantly up-regulated in patients having stable disease or who exhibited a partial response. Thus, the use of IL-7 as an adjuvant to IL-10 therapy in such responsive patients may provide additional therapeutic benefit.

Moreover, as described further herein, PEG-IL-10 treatment at therapeutically effective doses induced up-regulation of several inflammatory cytokines, including IL-7, GM-CSF, IL-4 and IFNγ. In particular, IL-7 was significantly up-regulated in patients having disease stabilization or tumor shrinkage. Thus, serum concentration levels of IL-7 can be used as a biomarker to indicate that a therapeutic serum concentration of IL-10 has been achieved and/or that the PEG-IL-10 dose administered to achieve that serum concentration was efficacious. Other cytokines that are up-regulated in response to IL-10 therapy (e.g., GM-CSF, IL-4 and IFNγ) may also be useful as biomarkers.

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it can correspond to a sequence of a naturally occurring human polypeptide or nucleic acid molecule. In addition to the human polypeptides and the nucleic acid molecules which encode them, the present disclosure contemplates IL-10-related polypeptides and corresponding nucleic acid molecules from other species.

### Definitions

Unless otherwise indicated, the following terms are intended to have the meaning set forth below. Other terms are defined elsewhere throughout the specification.

The terms “patient” or “subject” are used interchangeably to refer to a human or a non-human animal (e.g., a mammal).

The terms “administration”, “administer” and the like, as they apply to, for example, a subject, cell, tissue, organ, or biological fluid, refer to contact of, for example, IL-10 or PEG-IL-10), a nucleic acid (e.g., a nucleic acid encoding native human IL-10); a pharmaceutical composition comprising the foregoing, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering IL-10 or a pharmaceutical composition comprising IL-10) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. The terms may also be used in other contexts, such as situations where IL-10 or PEG-IL-10 contacts an IL-10 receptor in, for example, the fluid phase or colloidal phase.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering IL-10 or a pharmaceutical composition comprising IL-10) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based upon a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration can be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration of IL-10) or subjective parameter (e.g., a subject's feeling of well-being).

The term “small molecules” refers to chemical compounds having a molecular weight that is less than about 10 kDa, less than about 2 kDa, or less than about lkDa. Small molecules include, but are not limited to, inorganic molecules, organic molecules, organic molecules containing an inorganic component, molecules comprising a radioactive atom, and synthetic molecules. Therapeutically, a small molecule can be more permeable to cells, less susceptible to degradation, and less likely to elicit an immune response than large molecules.

The term “ligand” refers to, for example, peptide, polypeptide, membrane-associated or membrane-bound molecule, or complex thereof, that can act as an agonist or antagonist of a receptor. “Ligand” encompasses natural and synthetic ligands, e.g., cytokines, cytokine variants, analogs, muteins, and binding compositions derived from antibodies. “Ligand” also encompasses small molecules, e.g., peptide mimetics of cytokines and peptide mimetics of antibodies. The term also encompasses an agent that is neither an agonist nor antagonist, but that can bind to a receptor without significantly influencing its biological properties, e.g., signaling or adhesion. Moreover, the term includes a membrane-bound ligand that has been changed, e.g., by chemical or recombinant methods, to a soluble version of the membrane-bound ligand. A ligand or receptor can be entirely intracellular, that is, it can reside in the cytosol, nucleus, or some other intracellular compartment. The complex of a ligand and receptor is termed a “ligand-receptor complex.”

The terms “inhibitors” and “antagonists”, or “activators” and “agonists”, refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor can also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of an IL-10 agent (or the nucleic acid molecules encoding them), either directly or indirectly; or to enhance the ability of a molecule to produce an effect comparable to that of an IL-10 agent. The term “modulator” is meant to refer broadly to molecules that can effect the activities described above. By way of example, a modulator of, e.g., a gene, a receptor, a ligand, or a cell, is a molecule that alters an activity of the gene, receptor, ligand, or cell, where activity can be activated, inhibited, or altered in its regulatory properties. A modulator can act alone, or it can use a cofactor, e.g., a protein, metal ion, or small molecule. The term “modulator” includes agents that operate through the same mechanism of action as IL-10 (i.e., agents that modulate the same signaling pathway as IL-10 in a manner analogous thereto) and are capable of eliciting a biological response comparable to (or greater than) that of IL-10.

Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an Activator.

The “activity” of a molecule can describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term can also refer to activity in modulating or maintaining cell-to-cell interactions (e.g., adhesion), or activity in maintaining a structure of a cell (e.g., a cell membrane). “Activity” can also mean specific activity, e.g., [catalytic activity]/[mg protein], or [immunological activity]/[mg protein], concentration in a biological compartment, or the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect can refer to efficacy, stability, solubility, or immunogenicity.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; and the like.

It will be appreciated that throughout this disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

As used herein, the term “variant” encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Non-naturally-occurring variants include polypeptides and nucleic acids that comprise a change in amino acid or nucleotide sequence, respectively, where the change in sequence is artificially introduced (e.g., muteins); for example, the change is generated in the laboratory by human intervention (“hand of man”). Thus, herein a “mutein” refers broadly to mutated recombinant proteins that usually carry single or multiple amino acid substitutions and are frequently derived from cloned genes that have been subjected to site-directed or random mutagenesis, or from completely synthetic genes.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” an IL-10 polypeptide), is meant to indicate that the polypeptide or nucleic acid has a sequence that is based on that of a reference polypeptide or nucleic acid (e.g., a naturally occurring IL-10 polypeptide or an IL-10-encoding nucleic acid), and is not meant to be limiting as to the source or method in which the protein or nucleic acid is made. By way of example, the term “derived from” includes homologs or variants of reference amino acid or DNA sequences.

In the context of a polypeptide, the term “isolated” refers to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

“Enriched” means that a sample is non-naturally manipulated (e.g., by a scientist) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), or b) a concentration greater than the environment in which the polypeptide was made (e.g., as in a bacterial cell).

“Substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

The terms “specifically binds” or “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least ten times greater, at least 20-times greater, or at least 100-times greater than the affinity with any other antibody, or binding composition derived therefrom. In a particular embodiment, the antibody will have an affinity that is greater than about 10⁹ liters/mol, as determined by, e.g., Scatchard analysis (Munsen, et al. 1980 Analyt. Biochem. 107:220-239).

### IL-10 and PEG-IL-10

The anti-inflammatory cytokine IL-10, also known as human cytokine synthesis inhibitory factor (CSIF), is classified as a type(class)-2 cytokine, a set of cytokines that includes IL-19, IL-20, IL-22, IL-24 (Mda-7), and IL-26, interferons (IFN-α, -β, -γ, -δ, -ε, -κ, -Ω, and -τ) and interferon-like molecules (limitin, IL-28A, IL-28B, and IL-29).

IL-10 is a cytokine with pleiotropic effects in immunoregulation and inflammation. It is produced by mast cells, counteracting the inflammatory effect that these cells have at the site of an allergic reaction. While it is capable of inhibiting the synthesis of pro-inflammatory cytokines such as IFN-γ, IL-2, IL-3, TNFα and GM-CSF, IL-10 is also stimulatory towards certain T cells and mast cells and stimulates B-cell maturation, proliferation and antibody production. IL-10 can block NF-κB activity and is involved in the regulation of the JAK-STAT signaling pathway. It also induces the cytotoxic activity of CD8+ T-cells and the antibody production of B-cells, and it suppresses macrophage activity and tumor-promoting inflammation. The regulation of CD8+ T-cells is dose-dependent, wherein higher doses induce stronger cytotoxic responses.

Human IL-10 is a homodimer with a molecular mass of 37 kDa, wherein each 18.5 kDa monomer comprises 178 amino acids, the first 18 of which comprise a signal peptide, and two cysteine residues that form two intramolecular disulfide bonds. The IL-10 dimer becomes biologically inactive upon disruption of the non-covalent interactions between the two monomer subunits.

The present disclosure contemplates human IL-10 (NP_000563) and murine IL-10 (NP 034678), which exhibit 80% homology, and use thereof. In addition, the scope of the present disclosure includes IL-10 orthologs, and modified forms thereof, from other mammalian species, including rat (accession NP_036986.2; GI 148747382); cow (accession NP_776513.1; GI 41386772); sheep (accession NP_001009327.1; GI 57164347); dog (accession ABY86619.1; GI 166244598); and rabbit (accession AAC23839.1; GI 3242896).

As alluded to above, the terms “IL-10”, “IL-10 polypeptide(s), “IL-10 molecule(s)”, “IL-10 agent(s)” and the like are intended to be broadly construed and include, for example, human and non-human IL-10 -related polypeptides, including homologs, variants (including muteins), and fragments thereof, as well as IL-10 polypeptides having, for example, a leader sequence (e.g., the signal peptide), and modified versions of the foregoing. In further particular embodiments, IL-10, IL-10 polypeptide(s), and IL-10 agent(s) are agonists.

The IL-10 receptor, a type II cytokine receptor, consists of alpha and beta subunits, which are also referred to as R1 and R2, respectively. Receptor activation requires binding to both alpha and beta. One homodimer of an IL-10 polypeptide binds to alpha and the other homodimer of the same IL-10 polypeptide binds to beta.

The utility of recombinant human IL-10 is frequently limited by its relatively short serum half-life, which can be due to, for example, renal clearance, proteolytic degradation and monomerization in the blood stream. As a result, various approaches have been explored to improve the pharmacokinetic profile of IL-10 without disrupting its dimeric structure and thus adversely affecting its activity. Pegylation of IL-10 results in improvement of certain pharmacokinetic parameters (e.g., serum half-life) and/or enhancement of activity.

As used herein, the terms “pegylated IL-10” and “PEG-IL-10” refer to an IL-10 molecule having one or more polyethylene glycol molecules covalently attached to at least one amino acid residue of the IL-10 protein, generally via a linker, such that the attachment is stable. The terms “monopegylated IL-10” and “mono-PEG-IL-10” indicate that one polyethylene glycol molecule is covalently attached to a single amino acid residue on one subunit of the IL-10 dimer, generally via a linker. As used herein, the terms “dipegylated IL-10” and “di-PEG-IL-10” indicate that at least one polyethylene glycol molecule is attached to a single residue on each subunit of the IL-10 dimer, generally via a linker.

In certain embodiments, the PEG-IL-10 used in the present disclosure is a mono-PEG-IL-10 in which one to nine PEG molecules are covalently attached via a linker to the alpha amino group of the amino acid residue at the N-terminus of one subunit of the IL-10 dimer. Monopegylation on one IL-10 subunit generally results in a non-homogeneous mixture of non-pegylated, monopegylated and dipegylated IL-10 due to subunit shuffling. Moreover, allowing a pegylation reaction to proceed to completion will generally result in non-specific and multi-pegylated IL-10, thus reducing its bioactivity. Thus, particular embodiments of the present disclosure comprise the administration of a mixture of mono- and di-pegylated IL-10 produced by the methods described herein.

In particular embodiments, the average molecular weight of the PEG moiety is between about 5 kDa and about 50 kDa. Although the method or site of PEG attachment to IL-10 is not critical, in certain embodiments the pegylation does not alter, or only minimally alters, the activity of the IL-10 agent. In certain embodiments, the increase in half-life is greater than any decrease in biological activity. The biological activity of PEG-IL-10 is typically measured by assessing the levels of inflammatory cytokines (e.g., TNF-α or IFN-γ) in the serum of subjects challenged with a bacterial antigen (lipopolysaccharide (LPS)) and treated with PEG-IL-10, as described in U.S. Pat. No. 7,052,686.

IL-10 variants can be prepared with various objectives in mind, including increasing serum half-life, reducing an immune response against the IL-10, facilitating purification or preparation, decreasing conversion of IL-10 into its monomeric subunits, improving therapeutic efficacy, and lessening the severity or occurrence of side effects during therapeutic use. The amino acid sequence variants are usually predetermined variants not found in nature, although some can be post-translational variants, e.g., glycosylated variants. Any variant of IL-10 can be used provided it retains a suitable level of IL-10 activity.

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2) R, K; 3) F, Y, H, W, R; 4) G, A, T, S; 5) Q, N; and 6) D, E. Guidance for substitutions, insertions, or deletions can be based on alignments of amino acid sequences of different variant proteins or proteins from different species. Thus, in addition to any naturally-occurring IL-10 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution.

The present disclosure also contemplates active fragments (e.g., subsequences) of mature IL-10 containing contiguous amino acid residues derived from the mature IL-10. The length of contiguous amino acid residues of a peptide or a polypeptide subsequence varies depending on the specific naturally-occurring amino acid sequence from which the subsequence is derived. In general, peptides and polypeptides can be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

Additionally, IL-10 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a suitable IL-10 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

As discussed further below, the IL-10 polypeptides can be isolated from a natural source (e.g., an environment other than its naturally-occurring environment) and can also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides can also be synthetically produced (e.g., by cell-free chemical synthesis).

Nucleic acid molecules encoding the IL-10 agents are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-10 polypeptide due to degeneracy of the genetic code.

### Interleukin-7

IL-7, a hematopoietic growth factor that is primarily secreted by stromal cells in the bone marrow and thymus, is a cytokine that is important for B and T cell development. IL-7 stimulates the differentiation of pluripotent hematopoietic stem cells into lymphoid progenitor cells. It is also important for proliferation during certain stages of B-cell maturation, and for T cell and NK cell survival, development and homeostasis. IL-7 and the hepatocyte growth factor (HGF) form a heterodimer that functions as a pre-pro-B cell growth-stimulating factor.

IL-7 binds to the IL-7 receptor (IL-7R), a heterodimer consisting of the IL-7 receptor alpha chain (IL-7Ra or CD127) and the common gamma (yc or CD132) chain; both CD127 and CD132 are also components of other cytokine receptors. Binding of IL-7 to the IL-7R results in a cascade of signals important for T-cell development within the thymus and survival within the periphery. IL-7 signaling also plays an essential role in preventing apoptosis in developing thymocytes.

The IL-7R heterodimer is expressed at high levels on most mature T cells, and it is also expressed on dendritic cells and monocytes, on subsets of developing B cells and T cells, but not on mature B cells. Stimulation of IL-7R activates the Janus kinase/signal transducers and activators of transcription (JAK/STAT), src family kinases and phosphatidylinositol 3-kinase (PI3K-AKT) signaling pathways. The three-dimensional structure of IL-7 in complex with the ectodomain of the IL-7R has been determined using x-ray diffraction (McElroy CA, et al. (January 2009) Structure 17(1):54-56). Knock-out mice genetically lacking the IL-7R exhibit thymic atrophy, arrest of T-cell development, and severe lymphopenia, suggesting that IL-7 plays an essential role in lymphoid cell survival (Entrez Gene: IL7 interleukin 7).

As alluded to above, the terms “IL-7”, “IL-7 polypeptide(s)”, “IL-7 molecule(s)”, “IL-7 agent(s)” and the like are intended to be construed broadly and include, for example, human and non-human IL-7-related polypeptides, including homologs, variants (including muteins), and fragments thereof, as well as IL-7 polypeptides having, for example, a leader sequence, and modified versions of the foregoing. In further particular embodiments, the IL-7 agents are agonists. In certain embodiments, the IL-7 agent is mature human IL-10. In other embodiments, the IL-7 agent is a variant of mature human IL-7, wherein the variant exhibits activity comparable to the activity of mature human IL-7. The present disclosure also contemplates nucleic acid molecules encoding the foregoing.

IL-7 as an immunotherapy agent has been examined in several pre-clinical animal studies and in human clinical trials for various malignancies and during HIV infections (see, e.g., Fry TJ, et al. (June 2002) Blood 99(11):3892-904).

IL-7 was found to augment responses to immunization, a feature that makes IL-7 an attractive candidate for adjuvant therapy for potentiating cancer immunotherapies (see Capitini, C M, et al. (Aug 2009) J Intern Med 266(2):141-53). IL-7 could also be beneficial in improving immune recovery after allogenic stem cell transplant (Snyder K M, et al. (July 2006) Leuk Lymphoma 47(7):1222-28).

IL-7 may play a role in enhancing immune reconstitution in cancer patients following cytotoxic chemotherapy. Lymphopenia occurs commonly following dose-intensive cytotoxic regimens, and T cell counts, most notably CD4⁻ T cells, typically remain well below the normal range for months to years following chemotherapy (Mackall CL, et al. (1997) Blood 89:3700-07). IL-7 levels are increased in clinical conditions associated with lymphopenia, and strong inverse relationships have been observed between serum IL-7 levels and CD4⁺ counts in chemotherapy-induced lymphopenia, HIV-associated lymphopenia, idiopathic lymphopenia, following bone marrow transplantation, and following monoclonal antibody-mediated T cell-depleting therapy for autoimmune disease (Napolitano L A, et al. (January 2001) Nat Med 7(1):73-9). Given that IL-7 therapy enhances immune reconstitution and can augment even limited thymic function by facilitating peripheral expansion of recent thymic emigrants, IL-7 therapy could potentially repair the immune system of patients who have been depleted by cytotoxic chemotherapy (see, e.g., Fry T J, et al. (June 2002) Blood 99(11):3892-904).

Another strategy for the use of IL-7 in the context of anti-tumor therapy is to utilize IL-7 in adoptive immunotherapy, wherein cells are activated and expanded ex vivo, then infused back into the patient. Current technologies can readily expand T cells ex vivo 4-5 logs, and IL-7 is among the cytokines that can be used to sustain or optimize T cell expansion ex vivo (see Chen H-W, et al. (2006) Clinical Immunology 119:21-31). Systemic therapy with IL-7 may also be able to enhance survival of adoptively transferred T cells in vivo or augment other immune-based therapies (see, e.g., Powell DJ, et al. (2005) Blood 105:241-50).

Since IL-7 therapy potently enhances CD4⁺ T cell numbers, and because CD4+ T cell depletion is a hallmark of HIV infection, IL-7 offers promise as a candidate for therapeutic and vaccine adjuvant applications in HIV disease. When associated with antiretroviral therapy in HIV patients, IL-7 administration decreased local and systemic inflammation in patients that had incomplete T-cell reconstitution, suggesting a potential benefit of IL-7 therapy in this patient population (Sereti I, et al. (2014) Plos Path 10(1)).

### Methods and Models

The present disclosure contemplates various methods and models for identifying candidate subject populations (or individual subjects) having a disease, disorder or condition (e.g., an oncology-related disorder comprising a solid tumor) that can be responsive to the therapies described herein. Such therapies include monotherapy with an IL-10 agent (e.g., PEG-IL-10) and combination therapy with an IL-10 agent and an IL-7 agent. In some embodiments, the methods and models allow a determination of whether administration of the combination results in an additive or synergistic effect. In other embodiments, the methods and models allow a determination of whether administration of the combination results in fewer adverse effects.

Certain embodiments of the present disclosure comprise the use of in vitro, ex vivo and in vivo methods and/or models. The subject population (or individual subject) is a non-human animal (e.g., rodent) or human in certain embodiments of the present disclosure.

By way of example, but not limitation, one aspect of the present disclosure contemplates a method for determining whether a test subject having an oncology-, immune-, inflammatory-, or viral-related disease, disorder or condition described herein is a candidate for treatment with an of IL-10 agent (e.g., PEG-IL-10), the method comprising a) providing a test subject having an indicia of such a disease, disorder or condition, b) administering the IL-10 agent to the test subject in an amount sufficient to achieve a desired response in a reference population, and c) determining whether the test subject exhibits the desired response; wherein the determination of the desired response indicates that the test subject is a candidate for treatment. The skilled artisan is able to modify such methods for use with combination therapy, including an IL-10 agent in combination with an IL-7 agent.

The desired response can be any result deemed favorable under the circumstances. In some embodiments, the desired response is prevention of the progression of the oncology-, immune-, inflammatory-, or viral-related disease, disorder or condition, while in other embodiments the desired response is a regression or stabilization of one or more characteristics of the oncology-, immune-, inflammatory-, or viral-related disease, disorder or condition (e.g., reduction in tumor size). In still other embodiments, the desired response is reduction or elimination of one or more adverse effects.

As indicated above, the present disclosure also contemplates various models. Any model can be used that provides reliable, reproducible results. The skilled artisan is familiar with models that can be used in conjunction with the subject matter of the present disclosure; in one embodiment, the IL-10 agent (e.g., PEG-IL-10) is evaluated in a model comprising a non-human subject (e.g., a mouse). Particular embodiments of the present disclosure contemplate a model for determining whether an IL-10 agent, in combination with or without another agent, is a candidate for treating the oncology-, immune-, viral-, or inflammatory-related diseases, disorders or conditions described herein.

Further embodiments of the present disclosure comprise a method or model for determining the optimum amount of an IL-10 agent, in combination with or without another agent. An optimum amount can be, for example, an amount that achieves an optimal effect in a subject or subject population, or an amount that achieves a therapeutic effect while minimizing or eliminating the adverse effects associated with the therapeutic agent(s). By manipulating the amounts of the agent(s), a clinician is able to determine the optimal dosing regimen for treating an oncology-, immune-, inflammatory-, or viral-related disease, disorder or condition in an individual subject, or a population of subjects, or eliminating the adverse effects or reducing the adverse effects such that they are acceptable under the circumstances.

### Biomarkers

The present disclosure also contemplates the use of biomarkers in conjunction with the methods and models described herein. The term “biomarker(s)” refers to a characteristic that is objectively measured and evaluated as an indicator of normal biological processes, pathogenic processes, or pharmacologic responses to a therapeutic intervention. The indicator may be any substance, structure, or process that can be measured in the body or its products and influences or predicts the incidence of outcome or disease. A “biomarker” is often a laboratory measurement that reflects the activity of a disease process; by way of example, positron emission tomographic (PET) scanning of dopamine transporters may be used as a biomarker in Parkinson's disease.

As used herein, the terms “biomarker”, “surrogate marker”, “marker”, “surrogate endpoint” and the like may be used interchangeable or may have different meanings, depending on the context in which they are used. As noted above, a “biomarker” is generally defined as a laboratory measurement that reflects the activity of a disease process. Markers usually quantitatively correlate (either directly or inversely) with disease progression. In the clinical trial setting, a surrogate endpoint (or marker) is a measure of the effect of a specific treatment that may correlate with an actual clinical endpoint but does not necessarily have a precise relationship; that is, a surrogate endpoint can be viewed as a biomarker intended to substitute for a clinical endpoint. Surrogate endpoints can be obtained from different modalities, such as behavioral or cognitive scores, or biochemical biomarkers. The primary difference between a biomarker and a surrogate marker is that a biomarker is a “candidate” surrogate marker, whereas a surrogate marker is a test used and taken as a measure of the effects of a specific treatment. (See Katz, R. (2004) NeuroRx 1(2):189-95).

In some embodiments of the present disclosure, a biomarker(s) is used to predict a clinical response(s) to monotherapy with an IL-10 agent (e.g., PEG-IL-10). In some instances, a pre-treatment biomarker can be used in such therapy wherein the biomarker has been validated to the point at which it could be applied as part of standard-of-care therapeutic decision-making.

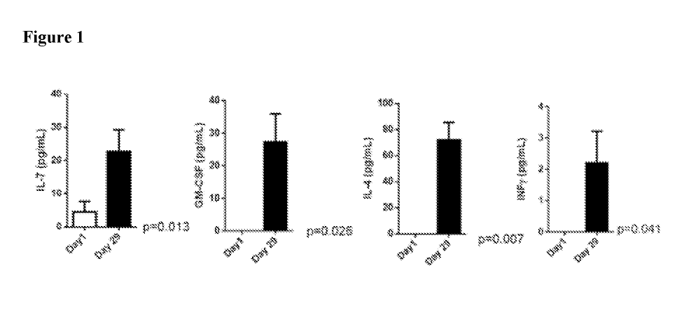

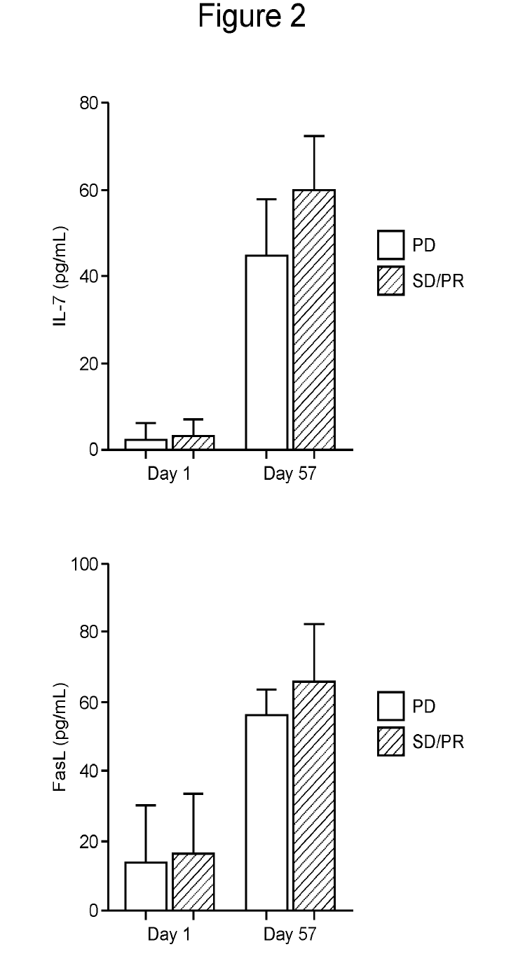

As described in the Experimental section, PEG-hIL-10 treatment at therapeutically effective doses induced up-regulation of the cytokines IL-7, GM-CSF, IL-4 and IFNy (see FIG. 1). IL-7 was significantly up-regulated in response to PEG-hIL-10 treatment, particularly in patients having disease stabilization (SD) or tumor shrinkage (PR) (see FIG. 2). Thus, serum concentration levels of IL-7 can be used as a biomarker indicating that a therapeutic serum concentration of IL-10 has been achieved and/or that the PEG-IL-10 dose administered to achieve that serum concentration was efficacious. Other cytokines that are up-regulated in response to IL-10 therapy (e.g., GM-CSF, IL-4 and IFNy) can also be used as biomarkers.

### Serum Concentrations

The blood plasma levels of IL-10 in the methods described herein can be characterized in several manners, including: (1) a mean IL-10 serum trough concentration above some specified level or in a range of levels; (2) a mean IL-10 serum trough concentration above some specified level for some amount of time; (3) a steady state IL-10 serum concentration level above or below some specified level or in a range of levels; or (4) a Cₘₐₓ of the concentration profile above or below some specified level or in some range of levels. As set forth herein, mean serum trough IL-10 concentrations have been found to be of particular import for efficacy in certain indications.

In some embodiments of the present disclosure, blood plasma and/or serum level concentration profiles that can be produced include: a mean IL-10 plasma and/or serum trough concentration of greater than about 1.0 pg/mL, greater than about 10.0 pg/mL, greater than about 20.0 pg/mL, greater than about 30 pg/mL, greater than about 40 pg/mL, greater than about 50.0 pg/mL, greater than about 60.0 pg/mL, greater than about 70.0 pg/mL, greater than about 80.0 pg/mL, greater than about 90 pg/mL, greater than about 0.1 ng/mL, greater than about 0.2 ng/mL, greater than about 0.3 ng/mL, greater than about 0.4 ng/mL, greater than about 0.5 ng/mL, greater than about 0.6 ng/mL, greater than about 0.7 ng/mL, greater than about 0.8 ng/mL, greater than about 0.9 ng/mL, greater than about 1.0 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, greater than about 5.0 ng/mL, greater than about 5.5 ng/mL, greater than about 6.0 ng/mL, greater than about 6.5 ng/mL, greater than about 7.0 ng/mL, greater than about 7.5 ng/mL, greater than about 8.0 ng/mL, greater than about 8.5 ng/mL, greater than about 9.0 ng/mL, greater than about 9.5 ng/mL, or greater than about 10.0 ng/mL.

In particular embodiments of the present disclosure, a mean IL-10 serum trough concentration is in the range of from 1.0 pg/mL to 10 ng/mL. In some embodiments, the mean IL-10 serum trough concentration is in the range of from 1.0 pg/mL to 100 pg/mL. In other embodiments, the mean IL-10 serum trough concentration is in the range of from 0.1 ng/mL to 1.0 ng/mL. In still other embodiments, the mean IL-10 serum trough concentration is in the range of from 1.0 ng/mL to 10 ng/mL. It is to be understood that the present disclosure contemplates ranges incorporating any concentrations encompassed by those set forth herein even if such ranges are not explicitly recited. By way of example, the mean serum IL-10 concentration in an embodiment can be in the range of from 0.5 ng/mL to 5 ng/mL. By way of further examples, particular embodiments of the present disclosure comprise a mean IL-10 serum trough concentration in a range of from about 0.5 ng/mL to about 10.5 ng/mL, from about 1.0 ng/mL to about 10.0 ng/mL, from about 1.0 ng/mL to about 9.0 ng/mL, from about 1.0 ng/mL to about 8.0 ng/mL, from about 1.0 ng/mL to about 7.0 ng/mL, from about 1.5 ng/mL to about 10.0 ng/mL, from about 1.5 ng/mL to about 9.0 ng/mL, from about 1.5 ng/mL to about 8.0 ng/mL, from about 1.5 ng/mL to about 7.0 ng/mL, from about 2.0 ng/mL to about 10.0 ng/mL, from about 2.0 ng/mL to about 9.0 ng/mL, from about 2.0 ng/mL to about 8.0 ng/mL, and from about 2.0 ng/mL to about 7.0 ng/mL.

In particular embodiments, a mean IL-10 serum trough concentration of 1-2 ng/mL is maintained over the duration of treatment. The present disclosure also contemplates embodiments wherein the mean IL-10 serum peak concentration is less than or equal to about 10.0 ng/mL over the duration of treatment. Further embodiments contemplate a mean IL-10 serum trough concentration greater than or equal to about 1.0 pg/mL. The optimal mean serum concentration is generally that at which the desired therapeutic effect is achieved without introducing undesired adverse effects.

Certain embodiments of the present disclosure provide a method for monitoring a subject receiving IL-10 therapy to predict, and thus potentially avoid, adverse effects, the method comprising: (1) measuring the subject's peak concentration of IL-10; (2) measuring the subject's trough concentration of IL-10; (3) calculating a peak-trough fluctuation; and, (4) using the calculated peak-trough fluctuation to predict potential adverse effects in the subject. In particular subject populations, a smaller peak-trough fluctuation indicates a lower probability that the subject will experience IL-10-related adverse effects. In addition, in some embodiments particular peak-trough fluctuations are determined for the treatment of particular diseases, disorders and conditions using particular dosing parameters, and those fluctuations are used as reference standards.

For the majority of drugs, plasma drug concentrations decline in a multi-exponential fashion. Immediately after intravenous administration, the drug rapidly distributes throughout an initial space (minimally defined as the plasma volume), and then a slower, equilibrative distribution to extravascular spaces (e.g., certain tissues) occurs. Intravenous IL-10 administration is associated with such a two-compartment kinetic model (see Rachmawati, H. et al. (2004) Pharm. Res. 21(11):2072-78). The pharmacokinetics of subcutaneous recombinant hIL-10 has also been studied (Radwanski, E. et al. (1998) Pharm. Res. 15(12):1895-1901). Thus, volume-of-distribution considerations are pertinent when assessing appropriate IL-10 dosing-related parameters. Moreover, efforts to target IL-10 agents to specific cell types have been explored (see, e.g., Rachmawati, H. (May 2007) Drug Met. Dist. 35(5):814-21), and the leveraging of IL-10 pharmacokinetic and dosing principles can prove invaluable to the success of such efforts.

The present disclosure contemplates administration of any dose and dosing regimen that results in maintenance of any of the IL-10 serum trough concentrations set forth above. By way of example, but not limitation, when the subject is a human, non-pegylated hIL-10 can be administered at a dose greater than 0.5 μg/kg/day, greater than 1.0 μg/kg/day, greater than 2.5 μg/kg/day, greater than 5 μg/kg/day, greater than 7.5 μg/kg, greater than 10.0 μg/kg, greater than 12.5 μg/kg, greater than 15 μg/kg/day, greater than 17.5 μg/kg/day, greater than 20 μg/kg/day, greater than 22.5 μg/kg/day, greater than 25 μg/kg/day, greater than 30 μg/kg/day, or greater than 35 μg/kg/day. In addition, by way of example, but not limitation, when the subject is a human, pegylated hIL-10 comprising a relatively small PEG (e.g., 5 kDa mono-di-PEG-hIL-10) can be administered at a dose greater than 0.5 μg/kg/day, greater than 0.75 μg/kg/day, greater than 1.0 μg/kg/day, greater than 1.25 μg/kg/day, greater than 1.5 μg/kg/day, greater than 1.75 μg/kg/day, greater than 2.0 μg/kg/day, greater than 2.25 μg/kg/day, greater than 2.5 μg/kg/day, greater than 2.75 μg/kg/day, greater than 3.0 μg/kg/day, greater than 3.25 μg/kg/day, greater than 3.5 μg/kg/day, greater than 3.75 μg/kg/day, greater than 4.0 μg/kg/day, greater than 4.25 μg/kg/day, greater than 4.5 μg/kg/day, greater than 4.75 μg/kg/day, or greater than 5.0 μg/kg/day.

Although the preceding discussion regarding IL-10 serum concentrations, doses and treatment protocols that are necessary to achieve particular IL-10 serum concentrations, etc., pertains to monotherapy with an IL-10 agent (e.g., PEG-IL-10), in certain embodiments such doses, treatment protocols, etc. are also relevant to therapeutic regimens comprising an IL-10 agent in combination with an IL-7 agent.

When an IL-10 agent (e.g., PEG-IL-10) is administered in combination with an IL-7 agent, one or more of the dosing parameters of the IL-10 agent applicable to monotherapy can be modified while the dosing parameters of the IL-7 agent applicable to monotherapy can remain the same; one or more of the dosing parameters of the IL-10 agent applicable to monotherapy can remain the same while one or more of the dosing parameters of the IL-7 agent applicable to monotherapy can be modified; one or more of the dosing parameters of the IL-10 agent and the IL-7 agent applicable to monotherapy can be modified; or the dosing parameters of each of the IL-10 agent and the IL-7 agent applicable to monotherapy can remain the same.

### Methods of Production of IL-10

A polypeptide of the present disclosure can be produced by any suitable method, including non-recombinant (e.g., chemical synthesis) and recombinant methods.

### A. Chemical Synthesis

Where a polypeptide is chemically synthesized, the synthesis can proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis can be performed as described hereafter. The alpha functions (Na) and any reactive side chains are protected with acid-labile or base-labile groups. The protective groups are stable under the conditions for linking amide bonds but can readily be cleaved without impairing the peptide chain that has formed. Suitable protective groups for the α-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), α,α-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy)valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40° C. and 60° C.) and with reaction times of, e.g., 2 to 72 hours.

The coupling of the Na-protected amino acid (e.g., the Fmoc amino acid) to the PAL, Wang or Rink anchor can, for example, be carried out with the aid of coupling reagents such as N,N′-di cyclohexylcarbodiimide (DCC), N,N′-diisopropylcarbodiimide (DIC) or other carbodiimides, 2-(1H-benzotriazol-1-yl)-1,1,3,3-tetramethyluronium tetrafluoroborate (TBTU) or other uronium salts, O-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10° C. and 50° C., for example, 25° C. in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Nα-Fmoc-amino acid, its acid chloride or acid fluoride, under the conditions described above.

The Nα-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Na-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10° C. and 50° C., e.g., at 25° C. The previously mentioned reagents for coupling the first Na-Fmoc amino acid to the PAL, Wang or Rink anchor are suitable as coupling reagents. Active esters of the protected amino acid, or chlorides or fluorides or symmetric anhydrides thereof can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5%-20% V/V of scavengers such as dimethylsulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within 0.5 to 3 hours, e.g., 2 hours. Peptides with fully protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10° C. and 50° C. (e.g., about 25° C.), and reaction times between about 12 and 24 hours (e.g., about 18 hours). In addition the peptide can be cleaved from the support by re-esterification, e.g., with methanol.

The acidic solution that is obtained can be admixed with a 3 to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

B. Recombinant Production

Methods describing the preparation of human and mouse IL-10 can be found in, for example, U.S. Patent No. 5,231,012, which teaches methods for the production of proteins having IL-10 activity, including recombinant and other synthetic techniques. IL-10 can be of viral origin, and the cloning and expression of a viral IL-10 from Epstein Barr virus (BCRF1 protein) is disclosed in Moore et al., (1990) Science 248:1230. IL-10 can be obtained in a number of ways using standard techniques known in the art, such as those described herein. Recombinant human IL-10 is also commercially available, e.g., from PeproTech, Inc., Rocky Hill, N.J.

Where a polypeptide is produced using recombinant techniques, the polypeptide can be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that can be used as host cells include insect cells, mammalian cells, and/or plant cells. Where mammalian host cells are used, they can include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide can be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences, and can provide for inducible or constitutive expression where the coding region is operably-linked under the transcriptional control of the transcriptional initiation region, and a transcriptional and translational termination region. In general, the transcriptional and translational regulatory sequences can include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Expression constructs generally have convenient restriction sites located near the promoter sequence to provide for the insertion of nucleic acid sequences encoding proteins of interest. A selectable marker operative in the expression host can be present to facilitate selection of cells containing the vector. Moreover, the expression construct can include additional elements. For example, the expression vector can have one or two replication systems, thus allowing it to be maintained in organisms, for example, in mammalian or insect cells for expression and in a prokaryotic host for cloning and amplification. In addition, the expression construct can contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein can be isolated using metal chelate chromatography methods. Proteins can contain modifications to facilitate isolation.

The polypeptides can be prepared in substantially pure or isolated form (e.g., free from other polypeptides). The polypeptides can be present in a composition that is enriched for the polypeptide relative to other components that can be present (e.g., other polypeptides or other host cell components). For example, purified polypeptide can be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%.

An IL-10 polypeptide can be generated using recombinant techniques to manipulate different IL-10-related nucleic acids known in the art to provide constructs capable of encoding the IL-10 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### Amide Bond Substitutions

In some cases, IL-10 includes one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of IL-10 can be substituted.

In another example, one or more amide linkages (—CO—NH—) in IL-10 can be replaced with a linkage which is an isostere of an amide linkage, such as —CH₂NH—, —CH₂S—, —CH₂CH₂—, —CH═CH-(cis and trans), —COCH₂—, —CH(OH)CH₂—or —CH₂SO—. One or more amide linkages in IL-10 can also be replaced by, for example, a reduced isostere pseudopeptide bond. See Couder et al. (1993) Int. J. Peptide Protein Res. 41:181-184. Such replacements and how to effect them are known to those of ordinary skill in the art.

### Amino Acid Substitutions

One or more amino acid substitutions can be made in an IL-10 polypeptide. The following are non-limiting examples:

a) substitution of alkyl-substituted hydrophobic amino acids, including alanine, leucine, isoleucine, valine, norleucine, (S)-2-aminobutyric acid, (5)-cyclohexylalanine or other simple alpha-amino acids substituted by an aliphatic side chain from C₁-C₁₀ carbons including branched, cyclic and straight chain alkyl, alkenyl or alkynyl substitutions;

b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid;

d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids;

e) substitution of side chain amide residues, including asparagine, glutamine, and alkyl or aromatic substituted derivatives of asparagine or glutamine; and

f) substitution of hydroxyl-containing amino acids, including serine, threonine, homoserine, 2,3-diaminopropionic acid, and alkyl or aromatic substituted derivatives of serine or threonine.

In some cases, IL-10 comprises one or more naturally occurring non-genetically encoded L-amino acids, synthetic L-amino acids, or D-enantiomers of an amino acid. For example, IL-10 can comprise only D-amino acids. For example, an IL-10 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, w-aminohexanoic acid, ω-aminoheptanoic acid, w-aminooctanoic acid, w-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

### Additional Modifications

A cysteine residue or a cysteine analog can be introduced into an IL-10 polypeptide to provide for linkage to another peptide via a disulfide linkage or to provide for cyclization of the IL-10 polypeptide. Methods of introducing a cysteine or cysteine analog are known in the art; see, e.g., U.S. Pat. No. 8,067,532.

An IL-10 polypeptide can be cyclized. One or more cysteines or cysteine analogs can be introduced into an IL-10 polypeptide, where the introduced cysteine or cysteine analog can form a disulfide bond with a second introduced cysteine or cysteine analog. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ-CO— or —(CH2)ₙ-C₆H₄—CO—) with functional groups which allow for the introduction of a bridge. Some examples are disulfides, disulfide mimetics such as the —(CH2)ₙ- carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (Ψv[CON]q), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

In some cases, one or more L-amino acids in an IL-10 polypeptide is replaced with one or more D-amino acids.

In some cases, an IL-10 polypeptide is a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids. [See, e.g., Jameson et al. (1994) Nature 368:744; and Brady et al. (1994) Nature 368:692].

An IL-10 polypeptide can include a “Protein Transduction Domain” (PTD), which refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-10 polypeptide, while in other embodiments, a PTD is covalently linked to the carboxyl terminus of an IL-10 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO:1); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:2); Transportan GWTLNSAGYLLGKINLKALAALAKKIL (SEQ ID NO:3); KALAWEAKLAKALAKALAKHLAKALAKALKCEA (SEQ ID NO:4); and RQIKIWFQNRRMKWKK (SEQ ID NO:5). Exemplary PTDs include, but are not limited to, YGRKKRRQRRR (SEQ ID NO:1), RKKRRQRRR (SEQ ID NO:6); an arginine homopolymer of from 3 arginine residues to 50 arginine residues; exemplary PTD domain amino acid sequences include, but are not limited to, any of the following: YGRKKRRQRRR (SEQ ID NO:1); RKKRRQRR (SEQ ID NO:7); YARAAARQARA (SEQ ID NO:8); THRLPRRRRRR (SEQ ID NO:9); and GGRRARRRRRR (SEQ ID NO:10).

The carboxyl group COR₃ of the amino acid at the C-terminal end of an IL-10 polypeptide can be present in a free form (R₃═OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C₁-C₆-alkyl alcohols, e.g., ethyl alcohol or tert-butanol. The carboxyl group can also be amidated with primary or secondary amines such as ammonia, branched or unbranched C₁-C₆-alkylamines or C₁-C₆ di-alkylamines, e.g., methylamine or dimethylamine.

The amino group of the amino acid NR₁R₂ at the N-terminus of an IL-10 polypeptide can be present in a free form (R₁═H and R₂═H) or in the form of a physiologically-tolerated salt such as, e.g., a chloride or acetate. The amino group can also be acetylated with acids such that R₁═H and R₂═acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂═C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

Particular Modifications to Enhance and/or Mimic IL-10 Function

It is frequently beneficial, and sometimes imperative, to improve one of more physical properties of the treatment modalities disclosed herein (e.g., IL-10) and/or the manner in which they are administered. Improvements of physical properties include, for example, modulating immunogenicity; methods of increasing water solubility, bioavailability, serum half-life, and/or therapeutic half-life; and/or modulating biological activity. Certain modifications can also be useful to, for example, raise of antibodies for use in detection assays (e.g., epitope tags) and to provide for ease of protein purification. Such improvements must generally be imparted without adversely impacting the bioactivity of the treatment modality and/or increasing its immunogenicity.

Pegylation of IL-10 is one particular modification contemplated by the present disclosure, while other modifications include, but are not limited to, glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins.

Pegylation: The clinical effectiveness of protein therapeutics is often limited by short plasma half-life and susceptibility to protease degradation. Studies of various therapeutic proteins (e.g., filgrastim) have shown that such difficulties can be overcome by various modifications, including conjugating or linking the polypeptide sequence to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

In addition to the beneficial effects of pegylation on pharmacokinetic parameters, pegylation itself can enhance activity. For example, PEG-IL-10 has been shown to be more efficacious against certain cancers than unpegylated IL-10 (see, e.g., EP 206636A2).

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, and examples are set forth elsewhere herein; by way of example, certain embodiments have molecular weights between 5 kDa and 20 kDa, while other embodiments have molecular weights between 4 kDa and 10 kDa.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography can be used to separate conjugates, and a fraction is then identified which contains the conjugate having, for example, the desired number of PEGs attached, purified free from unmodified protein sequences and from conjugates having other numbers of PEGs attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114; and Miron and Wilcheck (1993) Bio-conjug. Chem. 4:568-569) and benzotriazole carbonate PEG (BTC-PEG; see, e.g., Dolence, et al. US Patent No. 5,650,234), which react preferentially with lysine residues to form a carbamate linkage, but are also known to react with histidine and tyrosine residues. The linkage to histidine residues on certain molecules (e.g., IFNa) has been shown to be a hydrolytically unstable imidazolecarbamate linkage (see, e.g., Lee and McNemar, U.S. Pat. No. 5,985,263). Second generation pegylation technology has been designed to avoid these unstable linkages as well as the lack of selectivity in residue reactivity. Use of a PEG-aldehyde linker targets a single site on the N-terminus of a polypeptide through reductive amination.

PEG can be bound to a polypeptide of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide. Another activated polyethylene glycol which can be bound to a free amino group is 2,4-bis(O-methoxypolyethyleneglycol)-6-chloro-s-triazine, which can be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer can be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions can be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art can be used to terminate the reaction. In some embodiments the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686. Specific reaction conditions contemplated for use herein are set forth in the Experimental section.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Glycosylation: For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present.

Glycosylation can dramatically affect the physical properties (e.g., solubility) of polypeptides such as IL-10 and can also be important in protein stability, secretion, and subcellular localization. Glycosylated polypeptides can also exhibit enhanced stability or can improve one or more pharmacokinetic properties, such as half-life. In addition, solubility improvements can, for example, enable the generation of formulations more suitable for pharmaceutical administration than formulations comprising the non-glycosylated polypeptide.

Addition of glycosylation sites can be accomplished by altering the amino acid sequence. The alteration to the polypeptide can be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type can be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, can confer acidic properties to the glycoprotein. A particular embodiment of the present disclosure comprises the generation and use of N-glycosylation variants.

The polypeptide sequences of the present disclosure can optionally be altered through changes at the nucleic acid level, particularly by mutating the nucleic acid encoding the polypeptide at preselected bases such that codons are generated that will translate into the desired amino acids.

Polysialylation: The present disclosure also contemplates the use of polysialylation, the conjugation of polypeptides to the naturally occurring, biodegradable α-(2→8) linked polysialic acid (“PSA”) in order to improve the polypeptides' stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2):125-30). Various techniques for site-specific polysialylation are available (see, e.g., T. Lindhout et al., PNAS 108(18)7397-7402 (2011)).

Albumin Fusion: Additional suitable components and molecules for conjugation include albumins such as human serum albumin (HSA), cyno serum albumin, and bovine serum albumin (BSA).

According to the present disclosure, albumin can be conjugated to a drug molecule (e.g., a polypeptide described herein) at the carboxyl terminus, the amino terminus, both the carboxyl and amino termini, and internally (see, e.g., U.S. Pat. No. 5,876,969 and U.S. Pat. No. 7,056,701).

In the HSA—drug molecule conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a polypeptide drug molecule fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker, such as a peptide linker or modified version thereof

As alluded to above, fusion of albumin to one or more polypeptides of the present disclosure can, for example, be achieved by genetic manipulation, such that the nucleic acid coding for HSA, or a fragment thereof, is joined to the nucleic acid coding for the one or more polypeptide sequences.

Alternative Albumin Binding Strategies: Several albumin-binding strategies have been developed as alternatives to direct fusion and can be used with the IL-10 agents described herein. By way of example, the present disclosure contemplates albumin binding through a conjugated fatty acid chain (acylation) and fusion proteins which comprise an albumin binding domain (ABD) polypeptide sequence and the sequence of one or more of the polypeptides described herein.

Conjugation with Other Molecules: Additional suitable components and molecules for conjugation include, for example, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Thus, the present disclosure contemplates conjugation of one or more additional components or molecules at the N- and/or C-terminus of a polypeptide sequence, such as another polypeptide (e.g., a polypeptide having an amino acid sequence heterologous to the subject polypeptide), or a carrier molecule. Thus, an exemplary polypeptide sequence can be provided as a conjugate with another component or molecule.

An IL-10 polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria; and dendritic cells. Such conjugated forms can, if desired, be used to produce antibodies against a polypeptide of the present disclosure.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Fc-fusion Molecules: In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

Other Modifications: The present disclosure contemplates the use of other modifications, currently known or developed in the future, of IL-10 to improve one or more properties. Examples include hesylation, various aspects of which are described in, for example, U.S. Patent Appin. Nos. 2007/0134197 and 2006/0258607, and fusion molecules comprising SUMO as a fusion tag (LifeSensors, Inc.; Malvern, PA).

Linkers: Linkers and their use have been described above. Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Examples of flexible linkers include glycine polymers (G)ₙ, glycine-alanine polymers, alanine-serine polymers, glycine-serine polymers (for example, (GmSo)n, (GSGGS)ₙ (SEQ ID NO:11), (GₘSₒGₘ)ₙ, (GₘSₒGₘSₒGₘ)ₙ (SEQ ID NO:12), (GSGGSₘ)ₙ (SEQ ID NO:11), (GSGSₘG)ₙ (SEQ ID NO:12) and (GGGSₘ)ₙ (SEQ ID NO:13), and combinations thereof, where m, n, and o are each independently selected from an integer of at least 1 to 20, e.g., 1-18, 2-16, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Examples of flexible linkers include, but are not limited to GGSG (SEQ ID NO:14), GGSGG (SEQ ID NO:15), GSGSG (SEQ ID NO:12), GSGGG (SEQ ID NO:16), GGGSG (SEQ ID NO:17), and GSSSG (SEQ ID NO:18).

Additional examples of flexible linkers include glycine polymers (G)ₙ or glycine-serine polymers (e.g., (GS)ₙ, (GSGGS)ₙ (SEQ ID NO:11), (GGGS)ₙ (SEQ ID NO:13) and (GGGGS)ₙ (SEQ ID NO:19), where n=1 to 50, for example, 1, 2, 3, 4, 5, 6, 7, 8, 9, 10, 10-20, 20-30, 30-50). Exemplary flexible linkers include, but are not limited to GGGS (SEQ ID NO:15), GGGGS (SEQ ID NO:19), GGSG (SEQ ID NO:14), GGSGG (SEQ ID NO:15), GSGSG (SEQ ID NO:12), GSGGG (SEQ ID NO:16), GGGSG (SEQ ID NO:17), and GSSSG (SEQ ID NO:18). A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the Polypeptides disclosed herein. As described herein, the heterologous amino acid sequence may be a signal sequence and/or a fusion partner, such as, albumin, Fc sequence, and the like.

### Therapeutic and Prophylactic Uses

The present disclosure contemplates the use of the IL-10 agents described herein (e.g., PEG-IL-10) to treat or prevent diseases, disorders and/or conditions, and/or the symptoms thereof, as monotherapy or in combination therapy with an IL-7 agent. Although the therapeutic and prophylactic uses set forth hereafter are frequently described in the context of treatment with an IL-10 agent, it is to be understood that such uses are amenable to treatment comprising an IL-10 agent and an IL-7 agent.

While particular uses are described in detail hereafter, it is to be understood that the present disclosure is not so limited. Furthermore, although general categories of particular diseases, disorders and conditions are set forth hereafter, some of the diseases, disorders and conditions may be a member of more than one category, and others may not be a member of any of the disclosed categories.

Oncology-related Disorders. In accordance with the present disclosure, an IL-10 agent can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis. The disclosure contemplates reducing tolerance to a tumor cell or cancer cell antigen, e.g., by modulating activity of a regulatory T-cell and/or a CD8+ T-cell (see, e.g., Ramirez-Montagut, et al. (2003) Oncogene 22:3180-87; and Sawaya, et al. (2003) New Engl. J. Med. 349:1501-09). In particular embodiments, the tumor or cancer is colon cancer, ovarian cancer, breast cancer, melanoma, lung cancer, glioblastoma, or leukemia. The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the present disclosure provides methods for treating a proliferative condition, cancer, tumor, or precancerous condition with an IL-10 agent and at least one additional therapeutic or diagnostic agent, examples of which are set forth elsewhere herein.

Immune- and Inflammatory-related Disorders. As used herein, terms such as “immune disease”, “immune condition”, “immune disorder”, “inflammatory disease”, “inflammatory condition”, “inflammatory disorder” and the like are meant to broadly encompass any immune- or inflammatory-related condition (e.g., pathological inflammation and autoimmune diseases). Such conditions frequently are inextricably intertwined with other diseases, disorders and conditions. By way of example, an “immune condition” may refer to proliferative conditions, such as cancer, tumors, and angiogenesis; including infections (acute and chronic), tumors, and cancers that resist eradication by the immune system.

A non-limiting list of immune- and inflammatory-related diseases, disorders and conditions which may be treated or prevented with the compounds and compositions of the present disclosure include, arthritis (e.g., rheumatoid arthritis), kidney failure, lupus, asthma, psoriasis, colitis, pancreatitis, allergies, fibrosis, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (e.g., Crohn's disease and ulcerative colitis), allergic contact dermatitis and other eczemas, systemic sclerosis, transplantation and multiple sclerosis.

In some embodiments, an IL-10 agent described herein can be combined with an immunosuppressive agent to reduce the number of immune effector cells.

Viral-related Disorders. The present disclosure contemplates the use of the IL-10 agents in the treatment and/or prevention of any viral disease, disorder or condition for which treatment with an IL-10 agent may be beneficial. In particular embodiments, the viral disorder is a chronic viral disorder. Examples of viral diseases, disorders and conditions that are contemplated include, but are not limited to, hepatitis B virus (HBV), hepatitis C virus (HCV), human papilloma virus (HPV), HIV, AIDS (including its manifestations such as cachexia, dementia, and diarrhea), herpes simplex virus (HSV), Epstein-Barr virus (EBV), varicella zoster virus, coxsackie virus, and cytomegalovirus (CMV).

### Pharmaceutical Compositions

The IL-10 agents and other agents described herein (e.g., IL-7 agents) can be in the form of compositions suitable for administration to a subject. In general, such compositions are “pharmaceutical compositions” comprising IL-10 and/or another agent(s) described herein (e.g., an IL-7 agent), and one or more pharmaceutically acceptable or physiologically acceptable diluents, carriers or excipients. In certain embodiments, the IL-10 agents and other agent(s) are each present in a therapeutically acceptable amount. The pharmaceutical compositions can be used in the methods of the present disclosure; thus, for example, the pharmaceutical compositions can be administered ex vivo or in vivo to a subject in order to practice the therapeutic and prophylactic methods and uses described herein.

In the description of the pharmaceutical compositions, and aspects thereof, that follows, the pharmaceutical compositions are generally described in the context of an Il-10 agent (e.g., PEG-IL-10). However, it is to be understood that the description also applies to the other agent(s) described herein, either in pharmaceutical compositions comprising combinations of an IL-10 agent and another agent(s) (e.g., an IL-7 agent), or in pharmaceutical compositions comprising only one of the components.

The pharmaceutical compositions of the present disclosure can be formulated to be compatible with the intended method or route of administration; exemplary routes of administration are set forth herein. Furthermore, the pharmaceutical compositions can be used in combination with other therapeutically active agents or compounds as described herein in order to treat or prevent the diseases, disorders and conditions as contemplated by the present disclosure.

The pharmaceutical compositions typically comprise a therapeutically effective amount of an IL-10 agent contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle can be a physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it can be stored in sterile vials as a solution, suspension, gel, emulsion, solid, or dehydrated or lyophilized powder. Such formulations can be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus can be used to deliver IL-10, including implants (e.g., implantable pumps) and catheter systems, slow injection pumps and devices, all of which are well known to the skilled artisan. Depot injections, which are generally administered subcutaneously or intramuscularly, can also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions can be in the form of a sterile injectable aqueous or oleagenous suspension. This suspension can be formulated according to the known art using those suitable dispersing or wetting agents and suspending agents mentioned herein. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active ingredient can be in a form suitable for oral use, for example, as tablets, capsules, troches, lozenges, aqueous or oily suspensions, dispersible powders or granules, emulsions, hard or soft capsules, or syrups, solutions, microbeads or elixirs. In particular embodiments, an active ingredient of an agent co-administered with an IL-10 agent described herein is in a form suitable for oral use. Pharmaceutical compositions intended for oral use can be prepared according to any method known to the art for the manufacture of pharmaceutical compositions, and such compositions can contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients can be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration can be uncoated or coated by known techniques to delay disintegration and absorption in the gastrointestinal tract and thereby provide a sustained action. For example, a time-delay material such as glyceryl monostearate or glyceryl distearate can be employed. They can also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use can also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions can also contain one or more preservatives.

Oily suspensions can be formulated by suspending the active ingredient in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure can also be in the form of oil-in-water emulsions. The oily phase can be a vegetable oil, for example olive oil or arachis oil, or a mineral oil, for example, liquid paraffin, or mixtures of these. Suitable emulsifying agents can be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed.

The present disclosure contemplates the administration of the IL-10 polypeptides in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The IL-10 agents (e.g., PEG-IL-10) and other agents contemplated by the present disclosure can be in the form of any other suitable pharmaceutical composition (e.g., sprays for nasal or inhalation use) currently known or developed in the future.

The concentration of a polypeptide (e.g., IL-10) or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

### Routes of Administration

The present disclosure contemplates the administration of the IL-10 agents (e.g., PEG-IL-10) and the other agent(s) described herein (e.g., an IL-7 agent), and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, can also be utilized to release the IL-10 agents disclosed herein over a defined period of time.

In some particular embodiments of the present disclosure, the IL-10 agents (e.g., PEG-IL-10) and the other agents(s) (e.g., an IL-7 agent) are administered parenterally, and in further particular embodiments the parenteral administration is subcutaneous.

### Combination Therapy

The present disclosure contemplates the use of an IL-10 agent (e.g., PEG-IL-10) in combination with one or more active agents (e.g., chemotherapeutic agents) or other prophylactic or therapeutic modalities (e.g., radiation). In particular embodiments, the one or more active agents are an IL-7 agent(s), whereas in other embodiments the one or more active agents exhibit a desired therapeutic activity by a mechanism distinct from a cytokine. The present disclosure also contemplates the use of an IL-10 agent in combination with both an IL-7 agent(s) and an agent(s) that exhibits a desired therapeutic activity by a mechanism distinct from a cytokine. In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As set forth above, other prophylactic or therapeutic modalities may comprise radiation (e.g., localized radiation therapy or total body radiation therapy) and/or other treatment modalities of a non-pharmacological nature. The present disclosure contemplates treatment regimens wherein a radiation phase is preceded or followed by treatment with one or more additional agents as described herein. In some embodiments, the present disclosure further comprises the use of an IL-10 agent (e.g., PEG-IL-10) in combination with bone marrow transplantation, peripheral blood stem cell transplantation, or other types of transplantation therapy.

As used herein, “combination therapy” is meant to include therapies that can be administered separately, for example, formulated separately for separate administration (e.g., as may be provided in a kit), and therapies that can be administered together in a single formulation (i.e., a “co-formulation”).

In certain embodiments, the IL-10 agent and the other agent(s) (e.g., an IL-7 agent) are administered or applied sequentially, e.g., where one agent is administered prior to one or more other agents. In other embodiments, the IL-10 agent and the other agent(s) (e.g., an IL-7 agent) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

The IL-10 agents of the present disclosure may be used in combination with at least one other (active) agent in any manner appropriate under the circumstances. In one embodiment, treatment with the IL-10 agent and the other agent(s) (e.g., an IL-7 agent) is maintained over a period of time. In another embodiment, treatment with the at least one other agent(s) (e.g., an IL-7 agent) is reduced or discontinued (e.g., when the subject is stable), while treatment with an IL-10 agent of the present disclosure (e.g., PEG-IL-10) is maintained at a constant dosing regimen. In a further embodiment, treatment with the other agent(s) (e.g., an IL-7 agent) is reduced or discontinued (e.g., when the subject is stable), while treatment with an IL-10 agent of the present disclosure is reduced (e.g., lower dose, less frequent dosing or shorter treatment regimen). In yet another embodiment, treatment with the other agent(s) (e.g., an IL-7 agent) is reduced or discontinued (e.g., when the subject is stable), and treatment with the IL-10 agent of the present disclosure is increased (e.g., higher dose, more frequent dosing or longer treatment regimen). In yet another embodiment, treatment with the other agent(s) (e.g., an IL-7 agent) is maintained and treatment with the IL-10 agent of the present disclosure is reduced or discontinued (e.g., lower dose, less frequent dosing or shorter treatment regimen). In yet another embodiment, treatment with the other agent(s) (e.g., an IL-7 agent) and treatment with an IL-10 agent of the present disclosure (e.g., PEG-IL-10) are reduced or discontinued (e.g., lower dose, less frequent dosing or shorter treatment regimen).

Oncology-related Disorders. The present disclosure provides methods for treating and/or preventing a proliferative condition, cancer, tumor, or precancerous disease, disorder or condition with an IL-10 agent (e.g., PEG-IL-10) and at least one additional agent(s) (e.g., an IL-7 agent) or diagnostic agent exhibiting a desired activity.

In certain embodiments, the present disclosure provides methods for tumor suppression of tumor growth comprising administration of an IL-10 agent described herein in combination with a signal transduction inhibitor (STI) to achieve additive or synergistic suppression of tumor growth. As used herein, the term “signal transduction inhibitor” refers to an agent that selectively inhibits one or more steps in a signaling pathway. Signal transduction inhibitors (STIs) of the present disclosure include: (i) bcr/abl kinase inhibitors (e.g., GLEEVEC); (ii) epidermal growth factor (EGF) receptor inhibitors, including kinase inhibitors and antibodies; (iii) her-2/neu receptor inhibitors (e.g., HERCEPTIN); (iv) inhibitors of Akt family kinases or the Akt pathway (e.g., rapamycin); (v) cell cycle kinase inhibitors (e.g., flavopiridol); (vi) farnesyl transferase inhibitors (FTIs); and (vii) phosphatidyl inositol kinase inhibitors.

Agents involved in in immunomodulation can also be used in combination with the IL-10 agents described herein for the suppression of tumor growth in cancer patients. Suitable immunomodulators that may be used in the present disclosure include CD40L, B7, and B7RP1; activating monoclonal antibodies (mAbs) to stimulatory receptors, such as, ant-CD40, anti-CD38, anti-ICOS, and 4-IBB ligand; dendritic cell antigen loading (in vitro or in vivo); dendritic cell cancer vaccine; cytokines/chemokines, such as, IL-1, IL-2, IL-12, ELC/CCL19, SLC/CCL21, MCP-1, IL-4, IL-18, TNF, IL-15, MDC, IFNa/b, M-CSF, IL-3, GM-CSF, IL-13, and anti-IL-10; bacterial lipopolysaccharides (LPS); and immune-stimulatory oligonucleotides.

Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; and pharmaceutically acceptable salts, acids or derivatives of any of the above.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above. In certain embodiments, combination therapy comprises administration of a hormone or related hormonal agent.

Additional treatment modalities that may be used in combination with an IL-10 agent include a cytokine or cytokine antagonist, such as IL-12, INFa, or anti-epidermal growth factor receptor, radiotherapy, a monoclonal antibody against another tumor antigen, a complex of a monoclonal antibody and toxin, a T-cell adjuvant, bone marrow transplant, or antigen presenting cells (e.g., dendritic cell therapy). Vaccines (e.g., as a soluble protein or as a nucleic acid encoding the protein) are also provided herein.

The present disclosure encompasses pharmaceutically acceptable salts, acids or derivatives of any of the above.

Immune- and Inflammatory-related Disorders. The present disclosure provides methods for treating and/or preventing immune- and/or inflammatory-related diseases, disorders and conditions, as well as disorders associated therewith, with an IL-10 agent (e.g., PEG-IL-10) and at least one additional agent(s) (e.g., an IL-7 agent) or diagnostic agent exhibiting a desired activity.

Examples of therapeutic agents useful in combination therapy include, but are not limited to, the following: non-steroidal anti-inflammatory drug (NSAID) such as aspirin, ibuprofen, and other propionic acid derivatives (alminoprofen, benoxaprofen, bucloxic acid, carprofen, fenbufen, fenoprofen, fluprofen, flurbiprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone). Other combinations include cyclooxygenase-2 (COX-2) inhibitors.

Other active agents for combination include steroids such as prednisolone, prednisone, methylprednisolone, betamethasone, dexamethasone, or hydrocortisone. Such a combination may be especially advantageous since one or more adverse effects of the steroid can be reduced or even eliminated by tapering the steroid dose required.

Additional examples of active agents that may be used in combinations for treating, for example, rheumatoid arthritis, include cytokine suppressive anti-inflammatory drug(s) (CSAIDs); antibodies to, or antagonists of, other human cytokines or growth factors, for example, TNF, LT, IL-10, IL-2, IL-6, IL-7, IL-8, IL-15, IL-16, IL-18, EMAP-II, GM-CSF, FGF, or PDGF.

Particular combinations of active agents may interfere at different points in the autoimmune and subsequent inflammatory cascade, and include TNF antagonists such as chimeric, humanized or human TNF antibodies, REMICADE, anti-TNF antibody fragments (e.g., CDP870), and soluble p55 or p75 TNF receptors, derivatives thereof, p75TNFRIgG (ENBREL.) or p55TNFR1gG (LENERCEPT), soluble IL-13 receptor (sIL-13), and also TNFα-converting enzyme (TACE) inhibitors; similarly, IL-1 inhibitors (e.g., Interleukin-1-converting enzyme inhibitors) may be effective. Other combinations include Interleukin 11, anti-P7s and p-selectin glycoprotein ligand (PSGL). Other examples of agents useful in combination with the IL-10 agents described herein include interferon-β1a (AVONEX); interferon-β1b (BETASERON); copaxone; hyperbaric oxygen; intravenous immunoglobulin; clabribine; and antibodies to, or antagonists of, other human cytokines or growth factors (e.g., antibodies to CD40 ligand and CD80).

The present disclosure encompasses pharmaceutically acceptable salts, acids or derivatives of any of the above.

Immune Checkpoint Inhibitors. The present disclosure contemplates the use of an IL-10 agent (e.g., PEG-IL-10) and at least one additional agent(s) that is an immune checkpoint inhibitor. Such combinations may further comprise one or more additional agent(s) (e.g., an IL-7 agent) or diagnostic agent exhibiting a desired activity.

The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism.

T cells have been the major focus of efforts to therapeutically manipulate endogenous antitumor immunity because of i) their capacity for the selective recognition of peptides derived from proteins in all cellular compartments; ii) their capacity to directly recognize and kill antigen-expressing cells (by CD8+ effector T cells; also known as cytotoxic T lymphocytes (CTLs)); and iii) their ability to orchestrate diverse immune responses by CD4+ helper T cells, which integrate adaptive and innate effector mechanisms. In the clinical setting, the blockade of immune checkpoints—which results in the amplification of antigen-specific T cell responses—has shown to be a promising approach in human cancer therapeutics.

T cell-mediated immunity includes multiple sequential steps, each of which is regulated by counterbalancing stimulatory and inhibitory signals in order to optimize the response. While nearly all inhibitory signals in the immune response ultimately modulate intracellular signaling pathways, many are initiated through membrane receptors, the ligands of which are either membrane-bound or soluble (cytokines). While co-stimulatory and inhibitory receptors and ligands that regulate T-cell activation are frequently not over-expressed in cancers relative to normal tissues, inhibitory ligands and receptors that regulate T cell effector functions in tissues are commonly overexpressed on tumor cells or on non-transformed cells associated with the tumor microenvironment. The functions of the soluble and membrane-bound receptor-ligand immune checkpoints can be modulated using agonist antibodies (for co-stimulatory pathways) or antagonist antibodies (for inhibitory pathways). Thus, in contrast to most antibodies currently approved for cancer therapy, antibodies that block immune checkpoints do not target tumor cells directly, but rather target lymphocyte receptors or their ligands in order to enhance endogenous antitumor activity. [See Pardoll, (April 2012) Nature Rev. Cancer 12:252-64].

Examples of immune checkpoints (ligands and receptors), some of which are selectively up-regulated in various types of tumor cells, that are candidates for blockade include PD1 (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors, which can be divided into two classes based on their structural features: i) killer cell immunoglobulin-like receptors (KIRs), and ii) C-type lectin receptors (members of the type II transmembrane receptor family). Other less well-defined immune checkpoints have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

The present disclosure contemplates the use of an IL-10 agent (e.g., PEG-IL-10) and at least one additional agent(s) that is an inhibitor of the aforementioned immune-checkpoint receptors and ligands, as well as yet-to-be-described immune-checkpoint receptors and ligands. Such combinations may further comprise one or more additional agent(s) (e.g., an IL-7 agent) or diagnostic agent exhibiting a desired activity. Certain modulators of immune checkpoints are currently available, whereas others are in late-stage development. To illustrate, when it was approved for the treatment of melanoma in 2011, the fully humanized CTLA4 monoclonal antibody ipilimumab (YERVOY; Bristol-Myers Squibb) became the first immune checkpoint inhibitor to receive regulatory approval in the US. Fusion proteins comprising CTLA4 and an antibody (CTLA4-Ig; abatcept (ORENCIA; Bristol-Myers Squibb)) have been used for the treatment of rheumatoid arthritis, and other fusion proteins have been shown to be effective in renal transplantation patients that are sensitized to Epstein Barr Virus. PD1 antibodies are under development (e.g., nivolumab (Bristol-Myers Squibb) and lambrolizumab (Merck)), and anti-PDL1 antibodies are also being evaluated (e.g., MPDL3280A (Roche)). Nivolumab has shown promise in patients with melanoma, lung and kidney cancer.

The present disclosure encompasses pharmaceutically acceptable salts, acids or derivatives of any of the above.

Viral Diseases. The present disclosure provides methods for treating and/or preventing viral diseases, disorders and conditions, as well as disorders associated therewith, with an IL-10 agent (e.g., PEG-IL-10) and at least one additional agent(s) (e.g., an IL-7 agent) or diagnostic agent exhibiting a desired activity (e.g., one or more other antiviral agents and/or one or more agents not associated with viral therapy). In a particular embodiment, the viral disease is HIV.

Such combination therapy includes anti-viral agents targeting various viral life-cycle stages and having different mechanisms of action, including, but not limiting to, the following: inhibitors of viral uncoating (e.g., amantadine and rimantidine); reverse transcriptase inhibitors (e.g., acyclovir, zidovudine, and lamivudine); agents that target integrase; agents that block attachment of transcription factors to viral DNA; agents (e.g., antisense molecules) that impact translation (e.g., fomivirsen); agents that modulate translation/ribozyme function; protease inhibitors; viral assembly modulators (e.g., rifampicin); antiretrovirals such as, for example, nucleoside analogue reverse transcriptase inhibitors (e.g., azidothymidine (AZT), ddl, ddC, 3TC, d4T); non-nucleoside reverse transcriptase inhibitors (e.g., efavirenz, nevirapine); nucleotide analogue reverse transcriptase inhibitors; and agents that prevent release of viral particles (e.g., zanamivir and oseltamivir). Treatment and/or prevention of certain viral infections (e.g., HIV) frequently entail a group (“cocktail”) of antiviral agents.

Other antiviral agents contemplated for use in combination with an IL-10 agent include, but are not limited to, the following: abacavir, adefovir, amantadine, amprenavir, ampligen, arbidol, atazanavir, atripla, boceprevirertet, cidofovir, combivir, darunavir, delavirdine, didanosine, docosanol, edoxudine, emtricitabine, enfuvirtide, entecavir, famciclovir, fosamprenavir, foscarnet, fosfonet, ganciclovir, ibacitabine, imunovir, idoxuridine, imiquimod, indinavir, inosine, various interferons (e.g., peginterferon alfa-2a), lopinavir, loviride, maraviroc, moroxydine, methisazone, nelfinavir, nexavir, penciclovir, peramivir, pleconaril, podophyllotoxin, raltegravir, ribavirin, ritonavir, pyramidine, saquinavir, stavudine, telaprevir, tenofovir, tipranavir, trifluridine, trizivir, tromantadine, truvada, valaciclovir, valganciclovir, vicriviroc, vidarabine, viramidine, and zalcitabine.

The present disclosure encompasses pharmaceutically acceptable salts, acids or derivatives of any of the above.

### Dosing

The IL-10 agents (e.g., PEG-IL-10) of the present disclosure can be administered to a subject in an amount that is dependent upon, for example, the goal of the administration (e.g., the degree of resolution desired); the age, weight, sex, and health and physical condition of the subject; the formulation being administered; the route of administration; and the nature of the disease, disorder, condition or symptom thereof. The dosing regimen can also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

As discussed in detail elsewhere, the present disclosure contemplates administration of IL-10 to achieve certain serum trough concentrations and/or maintain certain mean serum trough concentrations.

In general, dosing parameters dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount can be more than the calculated ED50, in other situations the effective amount can be less than the calculated ED50, and in still other situations the effective amount can be the same as the calculated ED50.

In addition, an effective dose of the IL-10 agents (PEG-IL-10) of the present disclosure can be an amount that, when administered in one or more doses to a subject, produces a desired result relative to a healthy subject. For example, for a subject experiencing a particular disorder, an effective dose can be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

The amount of an IL-10 agent (e.g., PEG-IL-10) necessary to treat a disease, disorder or condition described herein is based on the IL-10 activity of the conjugated protein, which can be determined by IL-10 activity assays known in the art. By way of example, in the tumor context, suitable IL-10 activity includes, for example, CD8+ T-cell infiltrate into tumor sites, expression of inflammatory cytokines, such as IFN-γ, IL-4, IL-6, IL-10, and RANK-L, from these infiltrating cells, and increased levels of TNF-α or IFN-γ in biological samples.

In some particular embodiments of the present disclosure, the therapeutically effective amount of PEG-IL-10 can range from about 0.01 to100 μg protein/kg of body weight/day, from about 0.1 to 75 μg protein/kg of body weight/day, from about 0.5 to 50 μg protein/kg of body weight/day, or from about 20 to 40 μg protein/kg of body weight/day. In other particular embodiments, the therapeutically effective amount of PEG-IL-10 can range from about 0.01 to 100 μg protein/kg of body weight/day, from about 0.1 to 20 μg protein/kg of body weight/day, from about 0.5 to 10 μg protein/kg of body weight/day, or from about 1 to 4μg protein/kg of body weight/day.

In some embodiments, PEG-IL-10 is administered by continuous infusion to delivery about 1 to 800 μg protein/kg of body weight/day (e.g., about 1 to 16 μg protein/kg of body weight/day of PEG-IL-10). The infusion rate can be varied based on evaluation of, for example, adverse effects and blood cell counts. Other specific dosing parameters for the IL-10 agents are described elsewhere herein.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the disclosed IL-10 agent is contained in a “unit dosage form”. The phrase “unit dosage form” refers to physically discrete units, each unit containing a predetermined amount of the IL-10 agent of the present disclosure, either alone or in combination with one or more additional agents, sufficient to produce the desired effect. It will be appreciated that the parameters of a unit dosage form will depend on the particular agent and the effect to be achieved.

Although the description of dosing parameters, etc. set forth herein is presented in the context of an IL-10 agent, many aspects of the description are largely applicable to other agents (e.g., an IL-7 agent) contemplated for use in the combination therapy disclosed herein. Specific dosing parameters pertinent to such other agents can readily be ascertained from other sources, such as package inserts that accompany finished products for sale. In addition, the skilled artisan (e.g., a clinician) is able to apply her expertise to develop dosing parameters relevant to the therapies described herein.

### Kits

The present disclosure also contemplates kits comprising an IL-10 agent (e.g., PEG-IL-10), and a pharmaceutical composition thereof. The kits are generally in the form of a physical structure housing various components, as described below, and can be utilized, for example, in practicing the methods described above.

A kit can include an IL-10 agent (e.g., PEG-IL-10) disclosed herein (provided in, e.g., a sterile container), which can be in the form of a pharmaceutical composition suitable for administration to a subject. The IL-10 agent can be provided in a form that is ready for use or in a form requiring, for example, reconstitution or dilution prior to administration. When the IL-10 agent is in a form that needs to be reconstituted by a user, the kit can also include buffers, pharmaceutically acceptable excipients, and the like, packaged with or separately from the IL-10 agent. A kit can also contain both the IL-10 agent and/or an IL-7 agent(s) and/or another agent(s) as described herein; the kit can contain the several agents separately or they can already be combined in the kit. A kit of the present disclosure can be designed for conditions necessary to properly maintain the components housed therein (e.g., refrigeration or freezing).

A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.). Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, are provided.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that can be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: s or sec=second(s); min=minute(s); h or hr=hour(s); aa=amino acid(s); bp=base pair(s); kb=kilobase(s); nt =nucleotide(s); ng=nanogram; μg=microgram; mg=milligram; g=gram; kg=kilogram; dl or dL=deciliter; μl or μL=microliter; ml or mL=milliliter; 1 or L=liter; nM=nanomolar; ρM=micromolar; mM=millimolar; M=molar; kDa=kilodalton; i.m.=intramuscular(ly); i.p.=intraperitoneal(ly); SC or SQ=subcutaneous(ly); HPLC=high performance liquid chromatography; BW=body weight; U=unit; ns=not statistically significant; PMA=Phorbol 12-myristate 13-acetate; PBS=phosphate-buffered saline; DMEM=Dulbeco's Modification of Eagle's Medium; PBMCs=primary peripheral blood mononuclear cells; FBS=fetal bovine serum; FCS=fetal calf serum; HEPES=4-(2-hydroxyethyl)-1-piperazineethanesulfonic acid; LPS=lipopolysaccharide; RPMI=Roswell Park Memorial Institute medium; APC=antigen presenting cells; RCC=renal cell carcinoma; CRC=colorectal cancer; NSCLC=non-small cell lung cancer.

### Materials and Methods.

The following general materials and methods were used, where indicated, or may be used in the Examples below:

Molecular Biology Procedures. Standard methods in molecular biology are described in the scientific literature (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

Antibody-related Processes. Production, purification, and fragmentation of polyclonal and monoclonal antibodies are described (e.g., Harlow and Lane (1999) Using Antibodies, Cold Spring Harbor Laboratory Press, Cold Spring Harbor, N.Y.); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, N.J.); and fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.). Further discussion of antibodies appears elsewhere herein.

Software. Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, Calif.); and DeCypher™ (TimeLogic Corp., Crystal Bay, Nev.).

Pegylation. Pegylated IL-10 as described herein may be synthesized by any means known to the skilled artisan. Exemplary synthetic schemes for producing mono-PEG-IL-10 and a mix of mono-/di-PEG-IL-10 have been described (see, e.g., U.S. Pat. No. 7,052,686; US Pat. Publn. No. 2011/0250163; WO 2010/077853). Particular embodiments of the present disclosure comprise a mix of selectively pegylated mono- and di-PEG-IL-10. In addition to leveraging her own skills in the production and use of PEGs (and other drug delivery technologies) suitable in the practice of the present disclosure, the skilled artisan is familiar with many commercial suppliers of PEG-related technologies (e.g., NOF America Corp (Irvine, Calif.) and Parchem (New Rochelle, N.Y.)).

Animals. Various mice and other animal strains known to the skilled artisan can be used in conjunction with the teachings of the present disclosure. For example, immunocompetent Balb/C or B-cell-deficient Balb/C mice can be obtained from The Jackson Lab., Bar Harbor, Me. and used in accordance with standard procedures (see, e.g., Martin et al (2001) Infect. Immun., 69(11):7067-73 and Compton et al. (2004) Comp. Med. 54(6):681-89).

IL-10 Concentrations. Serum IL-10 concentration levels and exposure levels can be determined by standard methods used in the art. For example, when the experimental subject is a mouse, a serum exposure level assay can be performed by collecting whole blood (˜50 μL/mouse) from mouse tail snips into plain capillary tubes, separating serum and blood cells by centrifugation, and determining IL-10 exposure levels by standard ELISA kits and techniques.

The assays described hereafter are representative, and not exclusionary.

PBMC and CD8+ T-cell Gene Expression Assay. The following protocol provides an exemplary assay to examine gene expression.

Human PBMCs can be isolated according to any standard protocol (see, e.g., Fuss et al. (2009) Current Protocols in Immunology, Unit 7.1, John Wiley, Inc., NY). 2.5 mL of PBMCs (at a cell density of 8 million cells/mL) can be cultured per well with complete RPMI, containing RPMI (Life Technologies; Carlsbad, Calif.), 10 mM HEPES (Life Technologies; Carlsbad, Calif.), 10% FCS (Hyclone Thermo Fisher Scientific; Waltham, Mass.) and Penicillin/Streptomycin cocktail (Life Technologies; Carlsbad, CA), in any standard tissue culture treated 6-well plate (BD; Franklin Lakes, N.J.). Human pegylated-IL-10 can be added to the wells at a final concentration of 100 ng/mL, followed by a 7-day incubation. CD8+ T-cells can be isolated from the PBMCs using Miltenyi Biotec's MACS cell separation technology according to the manufacturer's protocol (Miltenyi Biotec; Auburn, Calif.). RNA can be extracted and cDNA can be synthesized from the isolated CD8+ T-cells and the CD8+ T-cell depleted-PBMCs using Qiagen's RNeasy Kit and RT² First Strand Kit, respectively, following the manufacturer's instructions (Qiagen N.V.; Netherlands). Quantitative PCR can be performed on the cDNA template using the RT² SYBR Green qPCR Mastermix and primers from Qiagen according to the manufacturer's protocol.

PBMC and CD8+ T-cell Cytokine Secretion Assay. Activated primary human CD8+ T-cells secrete IFN-γ when treated with PEG-IL-10 and then with an anti-CD3 antibody. The following protocol provides an exemplary assay to examine cytokine secretion.

Human PBMCs can be isolated according to any standard protocol (see, e.g., Fuss et al. (2009) Current Protocols in Immunology, Unit 7.1, John Wiley, Inc., NY). 2.5 mL of PBMCs (at a cell density of 8 million cells/mL) can be cultured per well with complete RPMI, containing RPMI (Life Technologies; Carlsbad, Calif.), 10 mM HEPES (Life Technologies; Carlsbad, Calif.), 10% FCS (Hyclone Thermo Fisher Scientific; Waltham, Mass.) and Penicillin/Streptomycin cocktail (Life Technologies; Carlsbad, Calif.), in any standard tissue culture treated 6-well plate (BD; Franklin Lakes, N.J.). Human pegylated-IL-10 can be added to the wells at a final concentration of 100 ng/mL, followed by a 3-day incubation. CD8+ T-cells can be isolated from the PBMCs using Miltenyi Biotec's MACS cell separation technology according to the manufacture's protocol (Miltenyi Biotec; Auburn, Calif.). The isolated CD8+ T-cells can then be cultured with complete RPMI containing 1 μg/mL anti-CD3 antibody (Affymetrix eBioscience) in any standard tissue culture plate for 4 hours. After the 4-hour incubation, the media can be collected and assayed for IFN-γ using a commercial ELISA kit and following the manufacture's protocol (Affymetrix eBioscience).

TNFα Inhibition Assay. PMA-stimulation of U937 cells (lymphoblast human cell line from lung available from Sigma-Aldrich (#85011440); St. Louis, Mo.) causes the cells to secrete TNFα, and subsequent treatment of these TNFα-secreting cells with human IL-10 causes a decrease in TNFα secretion in a dose-dependent manner. An exemplary TNFα inhibition assay can be performed using the following protocol.

After culturing U937 cells in RMPI containing 10% FBS/FCS and antibiotics, plate 1×105, 90% viable U937 cells in 96-well flat bottom plates (any plasma-treated tissue culture plates (e.g., Nunc; Thermo Scientific, USA) can be used) in triplicate per condition. Plate cells to provide for the following conditions (all in at least triplicate; for ‘media alone’ the number of wells is doubled because one-half will be used for viability after incubation with 10 nM PMA): 5 ng/mL LPS alone; 5 ng/mL LPS+0.1 ng/mL rhIL-10; 5 ng/mL LPS+1 ng/mL rhIL-10; 5 ng/mL LPS+10 ng/mL rhIL-10; 5 ng/mL LPS+100 ng/mL rhIL-10; 5 ng/mL LPS+1000 ng/mL rhIL-10; 5 ng/mL LPS+0.1ng/mL PEG-rhIL-10; 5 ng/mL LPS+1 ng/mL PEG-rhIL-10; 5 ng/mL LPS+10 ng/mL PEG-rhIL-10; 5 ng/mL LPS+100 ng/mL PEG-rhIL-10; and 5 ng/mL LPS+1000 ng/mL PEG-rhIL-10. Expose each well to 10 nM PMA in 200 μL for 24 hours, culturing at 37° C. in 5% CO₂ incubator, after which time ˜90% of cells should be adherent. The three extra wells can be re-suspended, and the cells are counted to assess viability (>90% should be viable). Wash gently but thoroughly 3× with fresh, non-PMA—containing media, ensuring that cells are still in the wells. Add 100 μL per well of media containing the appropriate concentrations (2× as the volume will be diluted by 100%) of rhIL-10 or PEG-rhIL-10, incubate at 37° C. in a 5% CO₂ incubator for 30 minutes. Add 100 μL per well of 10 ng/mL stock LPS to achieve a final concentration of 5 ng/mL LPS in each well, and incubate at 37° C. in a 5% CO₂ incubator for 18-24 hours. Remove supernatant and perform TNFα ELISA according to the manufacturer's instructions. Run each conditioned supernatant in duplicate in ELISA.

MC/9 Cell Proliferation Assay. IL-10 administration to MC/9 cells (murine cell line with characteristics of mast cells available from Cell Signaling Technology; Danvers, Mass.) causes increased cell proliferation in a dose-dependent manner. Thompson-Snipes, L. et al. (1991) J. Exp. Med. 173:507-10) describe a standard assay protocol in which MC/9 cells are supplemented with IL3+IL-10 and IL-3+IL-4+IL-10. Vendors (e.g., R&D Systems, USA; and Cell Signaling Technology, Danvers, Mass.) use the assay as a lot release assay for rhIL-10. Those of ordinary skill in the art will be able to modify the standard assay protocol described in Thompson-Snipes, L. et al, such that cells are only supplemented with IL-10.

Tumor Models and Tumor Analysis. Any art-accepted tumor model, assay, and the like can be used to evaluate the effect of the IL-10 agents described herein on various tumors. The tumor models and tumor analyses described hereafter are representative of those that can be utilized. Syngeneic mouse tumor cells are injected subcutaneously or intradermally at 10⁴, 10⁵ or 10⁶ cells per tumor inoculation. Ep2 mammary carcinoma, CT26 colon carcinoma, PDV6 squamous carcinoma of the skin and 4T1 breast carcinoma models can be used (see, e.g., Langowski et al. (2006) Nature 442:461-465). Immunocompetent Balb/C or B-cell deficient Balb/C mice can be used. PEG 10-mIL-10 can be administered to the immunocompetent mice, while PEG-hIL-10 treatment can be in the B-cell deficient mice. Tumors are allowed to reach a size of 100-250 mm³ before treatment is started. IL-10, PEG-mIL-10, PEG-hIL-10, or buffer control is administered SC at a site distant from the tumor implantation. Tumor growth is typically monitored twice weekly using electronic calipers. Tumor tissues and lymphatic organs are harvested at various endpoints to measure mRNA expression for a number of inflammatory markers and to perform immunohistochemistry for several inflammatory cell markers. The tissues are snap-frozen in liquid nitrogen and stored at −80° C. Primary tumor growth is typically monitored twice weekly using electronic calipers. Tumor volume can be calculated using the formula (width²×length/2) where length is the longer dimension. Tumors are allowed to reach a size of 90-250 mm³ before treatment is started.

### EXAMPLE 1

### Up-Regulation of IL-7 and Other Biomarkers

This example demonstrates that systemic immune activation, including up-regulation of Th1 cytokines and IL-7, occurred in all study patients.

A Phase I dose escalation study was conducted to evaluate the safety and tolerability of PEG-hIL-10 in patients with advanced solid tumors (RCC, melanoma, prostate, ovarian, pancreas, NSCLC and CRC). Thirty-three patients in dose-escalating cohorts of 4-6 patients received 1.0, 2.5, 5.0, 10, 20 and 40 μg/kg of PEG-hIL-10 SC daily. Patients continued on PEG-hIL-10 monotherapy until they met criteria for study discontinuation (significant adverse event(s) (AE), progressive disease (PD) or death). Patients could continue on PEG-IL-10 treatment with confirmed PD in the presence of clinical benefit and the absence of clinical deterioration.

Therapeutic efficacy was observed at PEG-hIL-10 doses of 20 μg/kg and 40 μg/kg, which correlated with median IL-10 serum concentrations of approximately more than 5,000 pg/mL. The concentrations of serum cytokines were measured using a multianalyte bead assay (Myriad RBM; Austin, Tex.) (and see Bea, J W et al. (May 2011) Clin Chem Lab Med 49(5):817-24) after 29 or 57 days in patients administered 20 μg/kg or 40 μg/kg PEG-hIL-10.

PEG-hIL-10 treatment at therapeutically effective doses induced a comprehensive immune signature in all patients analyzed, as indicated by increases of Thl cytokines (IFNy and IL-18); dendritic cell stimulation (GM-CSF, IL-4); growth factor for memory CD8⁺ T cells (IL-7); and CD8⁺ T cell activity (FasL). In particular, significant up-regulation of the cytokines IL-7, GM-CSF, IL-4 and IFNγ was observed following 29 days of PEG-hIL-10 therapy (see FIG. 1). These data indicate that these cytokines can serve as biomarkers to determine whether a therapeutically effective amount of PEG-hIL-10 has been administered.

In addition, IL-7 was significantly up-regulated in response to 57 days of PEG-hIL-10 treatment, particularly in patients having disease stabilization (SD) or tumor shrinkage (PR); IL-7 was also up-regulated, to a lower extent, in patients receiving a therapeutic dose who had progressive disease (PD) (see FIG. 2). These data indicate that administration of an IL-7 agent in combination with an IL-10 agent may be more efficacious than use of an IL-10 agent as monotherapy. Furthermore, after 57 days of PEG-hIL-10 treatment, FasL concentrations increased in patients exhibiting SD, PR and PD in a manner qualitatively similar to that observed with IL-7 (see FIG. 2). These data suggest that FasL can serve as a biomarker for T cell activation.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

1. A method of treating or preventing a disease, disorder or condition in a subject, comprising administering to the subject:
a) a therapeutically effective amount of an IL-7 agent, and
b) a therapeutically effective amount of an IL-10 agent; and
wherein the disease disorder or condition is a cancer-related disease, disorder or condition; an immune-related disease, disorder or condition; or a viral-related disease, disorder or condition.

2. A method of treating or preventing a disease, disorder or condition in a subject, comprising administering to the subject:
a) a therapeutically effective amount of an IL-7 agent, and
b) a therapeutically effective amount of an IL-10 agent, wherein the amount is sufficient to maintain a mean IL-10 serum trough concentration over a period of time of at least one week; and
wherein the mean IL-10 serum trough concentration is at least 1.0 ng/mL,
wherein the mean IL-10 serum trough concentration is maintained for at least 90% of the period of time, and
wherein the disease disorder or condition is a cancer-related disease, disorder or condition; an immune-related disease, disorder or condition; or a viral-related disease, disorder or condition.

3. The method of claim 2, wherein the mean IL-10 serum trough concentration is at least 1.5 ng/mL.

4. The method of claim 3, wherein the mean IL-10 serum trough concentration is at least 2.0 ng/ml.

5.-7. (canceled)

8. The method of claim 2, wherein the mean IL-10 serum trough concentration is maintained for at least 95% of the period of time.

9. The method of claim 8, wherein the mean IL-10 serum trough concentration is maintained for 100% of the period of time.

10. The method of claim 1, wherein the IL-10 agent is mature human IL-10.

11. The method of claim 1, wherein the IL-10 agent is a variant of mature human IL-10, and wherein the variant exhibits activity comparable to the activity of mature human IL-10.

12. The method of claim 1, wherein the disease, disorder or condition is a cancer-related disease, disorder or condition.

13. The method of claim 12, wherein the cancer-related disease, disorder or condition is a solid tumor or a hematological disorder.

14. The method of claim 12, wherein the cancer-related disease, disorder or condition is selected from the group consisting of melanoma, lung cancer, renal cancer colon cancer, head and neck cancer and breast cancer.

15. The method of claim 12, wherein the cancer-related disease, disorder or condition is leukemia or lymphoma.

16. The method of claim 1, wherein the disease, disorder or condition is an immune-related disease, disorder or condition.

17. The method of claim 1, wherein the disease, disorder or condition is a viral-related disease, disorder or condition.

18. The method of claim 1, wherein the IL-10 agent comprises at least one modification to form a modified IL-10 agent, wherein the modification does not alter the amino acid sequence of the IL-10 agent.

19. The method of claim 18, wherein the modified IL-10 agent is a PEG-IL-10 agent.

20. The method of claim 19, wherein the PEG-IL-10 agent comprises at least one PEG molecule covalently attached to at least one amino acid residue of at least one subunit of IL-10.

21. The method of claim 19, wherein the PEG-IL-10 agent comprises a mixture of mono-pegylated and di-pegylated IL-10.

22. The method of claim 19, wherein the PEG component of the PEG-IL-10 agent has a molecular mass from about 5 kDa to about 20 kDa.

23. The method of claim 19, wherein the PEG component of the PEG-IL-10 agent has a molecular mass greater than about 20 kDa.

24. The method of claim 19, wherein the modification comprises a linker.

25. The method of claim 18, wherein the modified IL-10 agent is an Fc fusion molecule.

26. The method of claim 18, wherein the modified IL-10 agent comprises a serum albumin or an albumin binding domain (ABD).

27. The method of claim 18, wherein the modified IL-10 agent is glycosylated or hesylated.

28. The method of claim 19, wherein the modification is site-specific.

29. The method of claim 1, wherein the administering of the IL-7 agent and the IL-10 agent is by parenteral injection.

30. The method of claim 29, wherein the administering of the IL-10 agent is by subcutaneous injection.

31. The method of claim 1, wherein the IL-7 agent and the IL-10 agent are administered simultaneously.

32. The method of claim 1, wherein the IL-7 agent and the IL-10 agent are administered sequentially.

33. The method of claim 1, further comprising administering at least one additional prophylactic or therapeutic agent.

34. The method of claim 33, wherein the prophylactic or therapeutic agent is a chemotherapeutic agent.

35. The method of claim 1, wherein the subject is a human.

36. A pharmaceutical composition, comprising
a) a therapeutically effective amount of an IL-7 agent of claim 1, and a pharmaceutically acceptable diluent, carrier or excipient; and
b) a therapeutically effective amount of an IL-10 agent of any onc of claims 1 35, and a pharmaceutically acceptable diluent, carrier or excipient.

37. The pharmaceutical composition of claim 36, wherein the excipient is an isotonic injection solution.

38. The pharmaceutical composition of claim 36 or 37, wherein the composition is suitable for human administration.

39. The pharmaceutical composition of claim 36, further comprising at least one additional prophylactic or therapeutic agent.

40. The pharmaceutical composition of claim 36, wherein the prophylactic or therapeutic agent is a chemotherapeutic agent.

41-88. (canceled)

89. The pharmaceutical composition of claim 36, wherein the IL-10 agent comprises a PEG-IL-10 agent.

90. The pharmaceutical composition of claim 89, wherein the IL-10 agent the PEG-IL-10 agent comprises a mixture of mono-pegylated and di-pegylated IL-10.

91. The method of claim 33, wherein the additional prophylactic or therapeutic agent is an immune checkpoint inhibitor.

92. The method of claim 91, wherein the immune checkpoint inhibitor is a monoclonal antibody.

93. The method of claim 92, wherein the monoclonal antibody is selected from the group consisting of anti-PD1 antibodies, anti-PDL1 antibodies, anti-LAG3 antibodies, anti-CTLA4 antibodies, anti-PDL1 antibodies, anti-IM3 antibodies.

94. The method of claim 93, wherein the anti-PD1 antibody is selected from the group consisting of nivolumab and lambrolizumab.
